(19) United States
(12) Patent Application Publication
(10) Pub. No.: US 2011/0045032 A1
(43) Pub. Date: Feb. 24, 2011

(54) COMPOSITION, FORMULATIONS & KIT FOR TREATMENT OF RESPIRATORY AND LUNG DISEASE WITH DEHYDROEPIANDROSTERONE(S) STEROID & AN ANTI-MUSCARINIC AGENT(S)

(75) Inventors: Cynthia B. Robinson, Wayne, PA (US); Jonathan W. Nyce, Titusville, NJ (US)

Correspondence Address: WILSON, SONSINI, GOODRICH & ROSATI, 650 PAGE MILL ROAD, PALO ALTO, CA, 94304-1050, US

(73) Assignee: EpiGenesis Pharmaceuticals, LLC, a Delaware Corporation, Cranbury, NJ (US)

(21) Appl. No.: 12/899,976

(22) Filed: Oct. 7, 2010

Related U.S. Application Data
(63) Continuation of application No. 10/461,563, filed on Jun. 12, 2003.
Application No. 10/461,563 is a continuation-in-part of application No. PCT/US02/12552, filed on Apr. 22, 2002.
(60) Provisional application No. 60/286,139, filed on Apr. 24, 2001.

(51) Int. Cl. A61K 31/5685; A61K 9/00; A61P 11/00; A61P 11/06

(52) U.S. Cl. 424/400; 514/171

(57) ABSTRACT

A pharmaceutical or veterinary composition comprises a non-corticosteroids, and/or salts thereof, and an anti-muscarinic (anti-cholinergic) agent, and/or pharmaceutically or veterinarily acceptable salts thereof. The composition is provided in various formulations and in the form of a kit. The products of this patent are useful in the prophylaxis and treatment of various respiratory, lung and malignant diseases.

### OTHER RELATED APPLICATIONS

This application is a continuation of U.S. Utility patent application Ser. No. 10/461,563, filed Jun. 12, 2003, which is a continuation-in-part of PCT Application No. PCT/US02/12552 (EPI-0449), entitled COMPOSITION, FORMULATIONS & KIT FOR TREATMENT OF RESPIRATORY & LUNG DISEASE WITH NON-GLUCOCORTICOID STEROIDS &/OR UBIQUINONE & BRONCHODILATING AGENT, filed Apr. 22, 2002, which is based on U.S. Provisional Application 60/286,139, filed Apr. 24, 2001, by Jonathan W. Nyce.

### BACKGROUND OF THE INVENTION

1. Field of the Invention

This invention relates to a composition and formulations comprising a dehydroepiandrosterone(s) of chemical formula (I), (II), (III), (IV) and (V), and/or salts thereof, and an anti-muscarinic receptor agent(s), and/or salts thereof, and optionally other bioactive agents and formulation components. These products are useful in the treatment of respiratory and lung diseases in general and in the treatment of conditions such as COPD, asthma, allergic rhinitis, and the like.

2. Description of the Background

Respiratory ailments are extremely common in the general population, and more so in certain ethnic groups, such as African Americans. In some cases they are accompanied by inflammation, which aggravates the condition of the lungs. Diseases such as Chronic obstructive pulmonary disease (COPD), asthma, allergic rhinitis, and Acute Respiratory Distress Syndrome (ARDS), including RDS in pregnant mothers and in premature born infants, among others, are common diseases in industrialized countries, and in the United States alone, they account for extremely high health care costs. These diseases have recently been increasing at an alarming rate, both in terms of prevalence, morbidity and mortality. In spite of this, their underlying causes still remain poorly understood. COPD is characterized by airflow obstruction that is generally caused by chronic bronchitis, emphysema, or both. Emphysema is characterized by abnormal permanent enlargement of the air spaces distal to the terminal bronchioles, accompanied by destruction of their walls and without obvious fibrosis. Chronic bronchitis is characterized by chronic cough, mucus production, or both, for at least three months for at least two successive years where other causes of chronic cough have been excluded. COPD characteristically affects middle aged and elderly people, and is one of the leading causes of morbidity and mortality worldwide. In the United States it affects about 14 million people and is the fourth leading cause of death, and both morbidity and mortality, have risen, for example, in the United States by 41% since 1982, and the age-adjusted death rates by 71% between 1966 and 1985. This contrasts with a decline over the same period in age-adjusted mortality from all causes (22%), and from cardiovascular diseases (45%). COPD, however, is preventable, given that its main cause is thought to be exposure to cigarette smoke. The disease is rare in lifetime non-smokers. Other proposed etiological factors include airway hyper-responsiveness or hypersensitivity, ambient air pollution, and allergy. The airflow obstruction in COPD is usually progressive in people who continue to smoke, and results in early disability and shortened survival time. Stopping smoking reverts the decline in lung function to values for non-smokers. Many COPD patients will use medication chronically for the rest of their lives, and will need increased doses and additional drugs during exacerbations. Amongst the currently available treatments for COPD, short-term benefits, but not long term effects, were found on its progression, from administration of anti-cholinergic drugs, β2 adrenergic agonists, and oral steroids. Neither anti-cholinergic drugs nor β2 adrenergic agonists have an effect on all people with COPD; nor do the two agents combined. The adverse effects of theophyllines and the need for frequent monitoring limit their usefulness. There is no evidence that anti-cholinergic agents affect the decline in lung function, and mucolytics have been shown to reduce the frequency of exacerbations but with a possible deleterious effect on lung function. The long-term effects of β2 adrenergic agonists, oral corticosteroids, and antibiotics have not yet been evaluated, and up to the present time no other drug has been shown to affect the progression of the disease or survival. Thus, there is very little currently available to alleviate symptoms of COPD, prevent exacerbations, preserve optimal lung function, and improve daily living activities and quality of life.

Asthma is a condition characterized by variable, in many instances reversible obstruction of the airways. This process is associated with lung inflammation and in some cases lung allergies. Many patients have acute episodes referred to as “asthma attacks,” while others are afflicted with a chronic condition. The asthmatic process may be triggered in some cases by inhalation of antigens by hypersensitive subjects. This condition is generally referred to as “extrinsic asthma”. Other asthmatics have an intrinsic predisposition to the condition, which is thus referred to as “intrinsic asthma”, and it encompasses conditions of different origin, including those mediated by the adenosine receptor(s), allergic conditions mediated by an immune IgE-mediated response, and others. All asthmas have a group of symptoms: bronchoconstriction, lung inflammation and/or decreased lung surfactant. Bronchodilators and anti-inflammatories are currently used in the treatment of asthma. Corticosteroids, the most common anti-inflammatories, have considerable side effects but are commonly prescribed nevertheless. Most of the drugs available for the treatment of asthma are, more importantly, barely effective in a small number of patients.

Acute Respiratory Distress Syndrome (ARDS), or stiff lung, shock lung, pump lung and congestive atelectasis, is believed to be caused by fluid accumulation within the lung which, in turn, causes the lung to stiffen. The condition is triggered within 48 hours by a variety of processes that injure the lungs such as trauma, head injury, shock, sepsis, multiple blood transfusions, medications, pulmonary embolism, severe pneumonia, smoke inhalation, radiation, high altitude, near drowning, and others. In general, ARDS occurs as a medical emergency and may be caused by other conditions that directly or indirectly cause the blood vessels to “leak” fluid into the lungs. In ARDS, the ability of the lungs to expand is severely decreased and produces extensive damage to the air sacs and lining or endothelium of the lung. ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. In some cases ARDS appears to be associated with other diseases, such as acute myelogenous leukemia, with acute tumor lysis syndrome (AILS) developed after treatment with, e.g. cytosine arabinoside. In general, however, ARDS is associated with traumatic injury, severe blood infections such as sepsis or other systemic illness, high dose radiation therapy, chemotherapy, and inflammatory responses that lead to multiple organ failure, and in many cases death. In premature babies (“premies”), the lungs are not quite developed and, therefore, the fetus is in an anoxic state during development. In addition, lung surfactant, a material critical for normal respiration, is generally not yet present in sufficient amounts at this early stage of life. Premies, however, often hyper-express the adenosine A₁ receptor and/or underexpress the adenosine A₂ₐ receptor, and are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

Although generally misdiagnosed, allergic rhinitis afflicts one in five Americans and occurs at all ages, thus accounting for an estimated $4 to 10 billion in health care costs each year. Symptoms include nasal congestion, discharge, sneezing, and itching, as well as itchy, watery, swollen eyes. Over time, allergic rhinitis sufferers often develop sinusitis, otitis media with effusion, and nasal polyposis, and may exacerbate asthma. It is associated also with mood and cognitive disturbances, fatigue and irritability. In allergic rhinitis, typically, IgE combines with allergens in the nose to produce chemical mediators, induction of cellular processes, and neurogenic stimulation, causing an underlying inflammation. Degranulation of mast cells results in the release of preformed mediators that interact with various cells, blood vessels, and mucous glands to produce the typical rhinitis symptoms. Most early- and late-phase reactions occur in the nose after allergen exposure. A late-phase reaction, however, is seen in chronic allergic rhinitis, accompanied with hypersecretion and congestion. Repeated exposure causes a hypersensitivity reaction to one or many allergens, and may also produce hyperreactivity to nonspecific triggers such as cold air or strong odors. Non-allergic rhinitis may be induced by infections, such as viruses, or associated with nasal polyps, as occurs in patients with aspirin idiosyncrasy, as well as by pregnancy, hypothyroidism, and exposure to occupational factors or medications. NARES syndrome, a non-allergic type of rhinitis associated with eosinophils in the nasal secretions, typically occurs in middle-aged individuals and is accompanied by loss of smell. Saline is often recommended to improve nasal stuffiness, sneezing, congestion, and mucosal irritation or dryness, minimize mucosal atrophy, and dislodge encrusted or thickened mucus, while causing no side effects, and may be tried first in pregnant patients. If used immediately before intranasal corticosteroid dosing, saline helps prevent local irritation. Anti-histamines often serve as a primary therapy. Terfenadine and astemizole, two non-sedating anti-histamines, however, have been associated with a ventricular arrhythmia known as Torsades de Points, usually in interaction with other medications such as ketoconazole and erythromycin, or secondary to an underlying cardiac problem. To date loratadine, another nonsedating anti-histamine, and cetirizine have not been associated with serious adverse cardiovascular events, the most common side effect of cetirizine being drowsiness. Claritin, for example, may be effective in relieving sneezing, runny nose, and nasal, ocular and palatal itching in a low percentage of patients, although not approved for this indication or asthma. Anti-histamines are typically combined with a decongestant to help relieve nasal congestion. Sympathomimetic medications are used as vasoconstrictors and decongestants, the three most common decongestants being pseudoephedrine, phenylpropanolamine and phenylephrine. These agents, however, cause hypertension, palpitations, tachycardia, restlessness, insomnia and headache. Anti-cholinergic agents, such as Cromolyn, have a role in patients with significant rhinorrhea or for specific entities such as “gustatory rhinitis”, which is usually associated with ingestion of spicy foods, and have been used on the common cold. Topical and nasal spray corticosteroids such as Vancenase are somewhat effective in the treatment of rhinitis, especially for symptoms of congestion, sneezing, and runny nose. Topical steroids are generally more effective than Cromolyn, particularly in the treatment of NARES, but side effects limit their usefulness except for temporary therapy in patients with severe symptoms. Immunotherapy, while expensive and inconvenient, often can provide substantial benefits, especially the use of drugs that produce blocking antibodies, alter cellular histamine release, and result in decreased IgE. Presently available treatments, such as propranolol, verapamil, and adenosine, may help to minimize symptoms. Verapamil is most commonly used but it has several shortcomings, since it causes or exacerbates systemic hypotension, congestive heart failure, bradyarrhythmias, and ventricular fibrillation. In addition, verapamil readily crosses the placenta and has been shown to cause fetal bradycardia, heart block, depression of contractility, and hypotension. Adenosine has several advantages over verapamil, including rapid onset, brevity of side effects, theoretical safety, and probable lack of placental transfer, but may not be administered to a variety of patients.

Pulmonary fibrosis, interstitial lung disease (ILD), or interstitial pulmonary fibrosis, include more than 130 chronic lung disorders that affect the lung by damaging lung tissue, and producing inflammation in the walls of the air sacs in the lung, scarring or fibrosis in the interstitium (or tissue between the air sacs), and stiffening of the lung, thus the name of the disease. Breathlessness during exercise may be one of the first symptoms of these diseases, and a dry cough may be present. Neither the symptoms nor X-rays are often sufficient to tell apart different types of pulmonary fibrosis. Some pulmonary fibrosis patients have known causes and some have unknown or idiopathic causes. The course of this disease is generally unpredictable. Its progression includes thickening and stiffening of the lung tissue, inflammation and difficult breathing. Some people may need oxygen therapy as part of their treatment.

Cancer is one of the most prevalent and feared diseases of our times. It generally results from the carcinogenic transformation of normal cells of different epithelia. Two of the most damaging characteristics of carcinomas and other types of malignancies are their uncontrolled growth and their ability to create metastases in distant sites of the host, particularly a human host. It is usually these distant metastases that may cause serious consequences to the host since, frequently, the primary carcinoma is removed by surgery. The treatment of cancer presently relies on surgery, irradiation therapy and systemic therapies such as chemotherapy, different immunity-boasting medicines and procedures, hyperthermia and systemic, radioactively labeled monoclonal antibody treatment, immunotoxins and chemotherapeutic drugs.

Steroid hormones are potent chemical messengers that exert dramatic effects on cell differentiation, homeostasis, and morphogenesis. These molecules diverse in structure share a mechanistically similar mode of action. The effector molecules diffuse across cellular membranes and bind to specific high affinity receptors in the target cell nuclei. This interaction results in the conversion of an inactive receptor to one that can interact with the regulatory regions of target genes and modulate the rate of transcription of specific gene sets. Upon ligand binding, these receptors generate both rapid and long lasting responses. Steroids can act through two basic mechanisms: genomic and non-genomic. The classical genomic action is mediated by specific intracellular receptors, whereas the primary target for the non-genomic one is the cell membrane. Many clinical symptoms seem to be mediated through the non-genomic route. Furthermore, membrane effects of steroid and other factors can interfere with the intranuclear receptor system inducing or repressing steroid- and receptor-specific genomic effects. These signalling pathways may lead to unexpected hormonal or anti-hormonal effects in patients treated with certain drugs. Steroid receptors are members of a large family of nuclear transcription factors that regulate gene expression by binding to their cognate steroid ligands, to the specific enhancer sequences of DNA (steroid response elements) and to the basic transcription machinery. Steroid receptors are basically localized in the nucleus, regardless of hormonal status, and considerable-amounts of unliganded steroid receptors may be present in the cytoplasm of target cells in exceptional cases. Most steroid receptors are phosphoproteins, which are further phosphorylated after ligand binding. The role of phosphorylation in receptor transaction is complex and may not be uniform to all steroid receptors. However, phosphorylation and/or dephosphorylation is believed to be a key event regulating the transcriptional activity of steroid receptors. Steroid receptor activities can be affected by the amount of steroid receptor in the cell nuclei, which is modified by the rate of transcription and translation of the steroid receptor gene as well as by proteolysis of the steroid receptor protein. There is an auto- and heteroregulation of receptor levels. Some of the steroid receptors appear to bind specific protease inhibitors and exhibit protease activity. Some steroid receptors are expressed as two or more isoforms, which may have different effects on transcription. Receptor isoforms are different translation or transcription products of a single gene. Isoform A of the progesterone receptor is a truncated form of PR isoform B originating from the same gene, but it is able to suppress not only the gene enhancing activity of PR-B but also that of other steroid receptors. Before hormone binding, the receptors are part of a complex with multiple chaperones which maintain the receptor in its steroid binding conformation. Following hormone binding, the complex dissociates and the receptors bind to steroid response elements in chromatin. Regulation of gene expression by hormones involves an interaction of the DNA-bound receptors with other sequence-specific transcription factors and with the general transcription factors, which is partly mediated by co-activators and co-repressors. The specific array of cis regulatory elements in a particular promoter/enhancer region, as well as the organization of the DNA sequences in nucleosomes, specifies the network of receptor interactions. Depending on the nature of these interactions, the final outcome can be induction or repression of transcription.

Dehydroepiandrosterone (DHEA) is a naturally occurring steroid secreted by the adrenal cortex with apparent chemoprotective properties. Epidemiological studies have shown that low endogenous levels of DHEA correlate with increased risk of developing some forms of cancer, such as pre-menopausal breast cancer in women and bladder cancer in both sexes. The ability of DHEA and DHEA analogues, such as DHEA-S sulfate, to inhibit carcinogenesis is believed to result from their uncompetitive inhibition of the activity of the enzyme glucose 6-phosphate dehydrogenase (G6PDH). DHEA, or 3β-hydroxyandrost-5-en-17-one or dehydroiso-androsterone, is a 17-ketosteroid which is quantitatively one of the major adrenocortical steroid hormones found in mammals. Clinically, DHEA has been used systemically and topically for treating psoriasis, gout, hyperlipemia, and it has been administered to post-coronary patients. DHEA has been shown also to have weight optimizing and anti-carcinogenic effects, and it has been used clinically in Europe in conjunction with estrogen as an agent to reverse menopausal symptoms and in the treatment of manic depression, schizophrenia, and Alzheimer's disease. DHEA has been used clinically at 40 mg/kg/day in the treatment of advanced cancer and multiple sclerosis. Side effects such as mild androgenic effects, hirsutism, and increased libido were observed and may be overcome by monitoring the dose and/or by using analogues. DHEA is used subcutaneously, orally, and as a patch to treat infections. DHEA is also a metabolic precursor of more powerful agents that increase immune response in mammals. DHEA is biphasic: it acts as an immuno-modulator when converted to androstenediol, androst-5-ene-30β,17β-diol (βAED), androstenetriol or androst-5-ene-3β,7β,17β-triol (βAET). Because of its lymphotoxic and suppressive effects on cell proliferation prior to its conversion to βAED and/or βAET, it is, believed that its superior immunity enhancing properties result from its conversion to more active metabolites. Dehydroepiandrosterone sulfate, (DHEA-S) has been shown to effectively attenuate eosinophilia and neutrophilia, as well as improving compliance and resistance, in three animal models of respiratory disease (mouse, rabbit, non-human primate). Chronic persistent asthma has been shown to be predominantly a neutrophil-driven disease (Gibson et al. (20-?), COPD has long been known to be neutrophil driven, and neutrophilia is observed in allergic rhinitis as well. Some patients receiving steroid hormones of adrenocortical origin at pharmacologically appropriate doses, however, show increased incidence of infectious disease. G6PDH is the rate limiting enzyme of the hexose monophosphate pathway, a major source of intracellular ribose-5-phosphate and NADPH. Ribose-5 phosphate is a necessary substrate for the synthesis of both ribo- and deoxyribonucleotides required for the synthesis of RNA and DNA. NADPH is a cofactor also involved in nucleic acid biosynthesis and the synthesis of hydroxmethylglutaryl Coenzyme A reductase (HMG CoA reductase). HMG CoA reductase is an unusual enzyme that requires two moles of NADPH for each mole of product, mevalonate, produced. Thus, it appears that HMG CoA reductase would be ultrasensitive to DHEA-mediated NADPH depletion, and that DHEA-treated cells would rapidly show the depletion of intracellular pools of mevalonate. Mevalonate is required for DNA synthesis, and DHEA arrests human cells in the G1 phase of the cell cycle in a manner closely resembling that of the direct HMG CoA. Because G6PDH produces mevalonic acid used in cellular processes such as protein isoprenylation and the synthesis of dolichol, a precursor for glycoprotein biosynthesis, DHEA inhibits carcinogenesis by depleting mevalonic acid and thereby inhibiting protein isoprenylation and glycoprotein synthesis. Mevalonate is the central precursor for the synthesis of cholesterol, as well as for the synthesis of a variety of non-sterol compounds involved in post-translational modification of proteins such as farnesyl pyrophosphate and geranyl pyrophosphate; for dolichol, which is required for the synthesis of glycoproteins involved in cell-to-cell communication and cell structure.

Inhaled anti-muscarinic agents are the treatment of choice, recommended by guidelines, in chronic obstructive pulmonary disease (COPD). In long-term clinical studies, ipratropium showed important effects beyond relaxation of airway smooth muscle, e.g. reduction of exacerbations of COPD. In phase III clinical trials the new generation anti-muscarinic tiotropium, inhaled once daily, has provided more than 24 hours of stable bronchodilation, that was sustained over the one-year treatment period. In addition, tiotropium in comparison to placebo and even ipratropium, has been shown to provide improvement in dyspnea, reduction of exacerbations of COPD, reduced hospital admissions for exacerbations, reduced duration of hospitalizations as well as improved health-related quality of life. Chronic effects, such as reduction of hospitalizations, are conventionally attributed to an anti-inflammatory action and not to symptomatic bronchodilation. The 24 hour stabilisation of airway patency, avoiding fluctuations of the diameter with occasional closure and consequent need for reopening, may explain the extended therapeutic profile of tiotropium. Inhibition by anti-muscarinics of pro-inflammatory cholinergic effects may also occur, e.g. inhibition of 5-HETE release from epithelial cells and inhibition of release of neutrophil and eosinophil chemotactic activity from alveolar macrophages. Anti-muscarinics agents have shown substantial value as a therapeutic approach in COPD.

A handful of medicaments have been used for the treatment of respiratory diseases, although they all have limitations. Amongst them are glucocorticoid steroids, leukotriene inhibitors, anti-cholinergic agents, anti-histamines, oxygen therapy, theophyllines, and mucolytics. Glucocorticoid steroids are the ones with the most widespread use in spite of their well documented side effects. Most of the available drugs are nevertheless effective in a small number of cases, and not at all when it comes to the treatment of asthma. No treatments are currently available for many of the other respiratory diseases. Theophylline, an important drug in the treatment of asthma, is a known adenosine receptor antagonist. Selective adenosine A1 receptor antagonists, 8-cyclopentyl-1,3-dipropylxanthine (DPCPX) and an anti-sense oligonucleotide were also reported to inhibit adenosine-mediated bronchoconstriction, inflammation and bronchial hyperresponsiveness in allergic rabbits. For many years, two classes of compounds have dominated the treatment of asthma: glucocorticosteroids and bronchodilators. Examples of glucocorticosteroids are beclomethasone and corticoid 21-sulfopropionates. Examples of a bronchodilator are an older β2 adrenergic agonist such as albuterol, and a newer one such as salmeterol. In general, when glucocorticosteroids are taken daily either by inhalation or orally, they attenuate inflammation. The β2 adrenergic agonists, on the other hand, primarily alleviate bronchoconstriction. Whereas glucocorticosteroids are not useful in general for acute settings, bronchodilators are used in acute care, such as in the case of asthma attacks. At the present time, many asthma patients require daily use of both types of agents, a glucocorticosteroid to contain pulmonary inflammation, and a bronchodilator to alleviate bronchoconstriction. More recently, fluticasone propionate, a glucocorticoid steroid was combined with β2 adrenergic agonists in one therapeutic formulation said to have greater efficiency in the treatment of asthma. However, glucocorticosteriods, particularly when taken for prolonged periods of time, have extremely deleterious side effects that, although somewhat effective, make their chronic use undesirable, particularly in children.

Clearly, there exists a well defined need for novel and effective therapies for treating respiratory, lung and cancer ailments that cannot presently be treated, or at least for which no therapies are available that are effective and devoid of significant detrimental side effects. This is the case of ailments afflicting the respiratory tract, and more particularly the lung and the lung airways, including respiratory difficulties, bronchoconstriction, lung inflammation and allergies, depletion or hyposecretion of surfactant, COPD, asthma, allergic rhinitis, etc. Moreover, there is a definite need for treatments that have prophylactic and therapeutic applications, and require low amounts of active agents, which makes them both less costly and less prone to detrimental side effects.

### SUMMARY OF THE INVENTION

The present invention relates to a composition, formulations and treatments employing a first active agent comprising a dehydroepiandrosterone(s) of chemical formula (I), (II), (III), (IV), and (V) and/or its salts in combination with a second active agent comprising an anti-muscarinic agent(s) and/or its salts, and optionally other bioactive agents including other types of anti-inflammatories and bronchodilating agents, and formulation ingredients. This composition and formulations are useful for treating lung and respiratory diseases and conditions such as Chronic Obstructive Pulmonary Disease (COPD), asthma, allergic rhinitis and many others that are associated with bronchoconstriction, lung inflammation and/or allergies as well as with changes in pulmonary surfaces.

The drawings accompanying this patent form part of the disclosure of the invention, and further illustrate some aspects of the present invention as discussed below.

### BRIEF DESCRIPTION OF THE DRAWINGS

FIG. 1 illustrates the inhibition of HT-29 SF cells by DHEA.

FIGS. 2a and 2b illustrates the effects of DHEA on cell cycle distribution in HT-29 SF cells.

FIGS. 3a and 3b illustrate the reversal of DHEA-induced growth inhibition in HT-29 cells.

FIGS. 4a, 4b, 4c, and 4d illustrates the reversal of DHEA-induced G₁ arrest in HT-29 SF cells.

### DETAILED DESCRIPTION OF TOE PREFERRED EMBODIMENTS

The present invention arose from a desire by the inventors to improve on prior treatments of respiratory and lung diseases, and other pathologies secondarily afflicting the lung. The present treatment is effective for treating a plurality of respiratory and lung diseases, whatever their cause, whether due to inhalation of tobacco components, other particulate matter or allergens, to steroid administration, abnormalities in adenosine or adenosine receptor metabolism or synthesis, or any other cause. The present invention provides a composition, formulations and a method for treating respiratory and lung diseases and conditions regardless of their mechanism. The present products are particularly suitable for treating diseases and conditions such as chronic obstructive pulmonary disease (COPD), asthma, allergic rhinitis, cystic fibrosis (CF), dyspnea, emphysema, wheezing, pulmonary hypertension, pulmonary fibrosis, hyper-responsive airways, increased adenosine or adenosine receptor levels, particularly those associated with infectious diseases, lung inflammation and/or allergy(ies), surfactant depletion, chronic bronchitis, bronchoconstriction, difficult breathing, impeded or obstructed lung airways, adenosine test for cardiac function, pulmonary vasoconstriction, impeded respiration, Acute Respiratory Distress Syndrome (ARDS), infantile Respiratory Distress Syndrome (infantile RDS), pain, decreased lung surfactant, or chronic bronchitis, among others.

There is very little currently available to alleviate symptoms of COPD, prevent exacerbations, preserve optimal lung function, and improve daily living activities and quality of life. Anti-cholinergic drugs generally achieve short-term bronchodilation and produce some symptom relief in people with COPD, but no improved long-term prognosis has been achieved when administered by themselves even when the products are inhaled. Most COPD patients have at least some measure of airways obstruction that is to some extent alleviated by ipratropium bromide alone. “The lung health study” found in men and women smokers spirometric signs of early COPD. Three treatments compared over a five-year period found that ipratropium bromide had no significant effect on the decline in the functional effective volume of the patient's lungs whereas smoking cessation produced a slowing of the decline in the functional effective volume of the lungs. Large amounts of ipratropium bromide are required when this drug is administered by itself, and at these doses it produces serious adverse effects, such as cardiac symptoms, hypertension, skin rashes, and urinary retention. Short and long acting inhaled β2 adrenergic agonists achieve short-term bronchodilation and provide some symptomatic relief in COPD patients, but show no meaningful maintenance effect on the progression of the disease. Short acting β2 adrenergic agonists improve symptoms in subjects with COPD, such as increasing exercise capacity and produce some degree of bronchodilation, and even an increase in lung function in some severe cases. The maximum effectiveness of the newer long acting inhaled β2 adrenergic agonists was found to be comparable to that of short acting β2 adrenergic agonists. Salmeterol was found to improve symptoms and quality of life, although only producing modest or no change in lung function. In asthmatics, however, β2 adrenergic agonists have been linked to an increased risk of death, worsened control of asthma, and deterioration in lung function. Continuous treatment of asthmatic and COPD patients with the bronchodilators ipratropium bromide or fenoterol resulted in a faster decline in lung function, when compared with treatment provided on a need basis, therefore indicating that they are not suitable for maintenance treatment. The most common immediate adverse effect of β2 adrenergic agonists, on the other hand, is tremors, which at high doses may cause a fall in plasma potassium, dysrhythmias, and reduced arterial oxygen tension. The combination of a β2 adrenergic agonist with an anti-cholinergic drug provides little additional bronchodilation compared with either drug alone. The addition of ipratropium to a standard dose of inhaled β2 adrenergic agonists for about 90 days, however, produces some improvement in stable COPD patients over either drug alone. Anti-cholinergic agents were found to produce greater bronchodilation than β2 adrenergic agonists in people with COPD. Ipratropium bromide given to patients without bronchodilator therapy produced an improvement of the functional effective volume of the patient's lungs that was greater when administered in conjunction with an anti-cholinergic agent than with a β2 adrenergic agonist, given the residual effect of the anti-cholinergic drug. Overall, the occurrence of adverse effects with β2 adrenergic agonists, such as tremor and dysrhythmias, is more frequent than with anti-cholinergics. Theophyllines have a small bronchodilatory effect in COPD patients whereas they have some common adverse effects, and they have a small therapeutic range given that blood concentrations of 15-20 mg/l are required for optimal effects. Adverse effects include nausea, diarrhea, headache, irritability, seizures, and cardiac arrhythmias, and they occur at highly variable blood concentrations and, in many people, they occur within the therapeutic range. The theophyllines' doses must be adjusted individually according to smoking habits, infection, and other treatments, which is cumbersome. Although theophyllines have been claimed to have an anti-inflammatory effect in asthma, especially at lower doses, none has been reported in COPD, although their bronchodilating short-term effect appears to be statistically different from placebo. Oral corticosteroids show some improvement in baseline functional effective volume in stable COPD patients whereas systemic corticosteroids have been found to be harmful at least producing some osteoporosis and inducing overt diabetes. The longer term use of oral corticosteroids may be useful in COPD, but it usefulness must be weighed against their substantial adverse effects. Inhaled corticosteroids have been found to have no real short-term effect in airway hyper-responsiveness to histamine, but a small long-term effect on lung function, e.g., in pre-bronchodilator functional effective volume. Fluticasone treatment of COPD patients showed a significant reduction in moderate and severe (but not mild) exacerbations, and a small but significant improvement in lung function and six minute walking distance. Oral prednisolone, inhaled beclomethasone or both had no effects in COPD patients, but lung function improved oral corticosteroids. Mucolytics have a modest beneficial effect on the frequency and duration of exacerbations but an adverse effect on lung function. Neither N-acetylcysteine nor other mucolytics, however, have a significant effect in people with severe COPD (functional effective volume <50%) in spite of evidencing greater reductions in frequency of exacerbation. N-acetylcysteine produced gastrointestinal side effect. Long-term oxygen therapy administered to hypoxaemic COPD and congestive cardiac failure patients, had little effect on their rates of death for the first 500 days or so, but survival rates in men increased afterwards and remained constant over the next five years. In women, however, oxygen decreased the rates of death throughout the study. Continuous oxygen treatment of hypoxemic COPD patients (functional effective volume <70% predicted) for 19.3 years decreased overall risk of death. To date, however, only life style changes, smoking cessation and long term treatment with oxygen (in hypoxaemics), have been found to alter the long-term course of COPD. Chronic obstructive pulmonary disease, (COPD), which affects approximately 14 million Americans, is the fourth leading cause of death in the United States and is responsible for an estimated US$6.5 billion in direct and indirect costs per year [1,2]. Its usual course is a slow deterioration of lung function and progressive breathlessness with activities. The age-adjusted death rate for COPD rose 71% from 1967 to 1987, and the year mortality rate is about 50%. Bronchodilators form one of the mainstays of therapy in COPD patients. The judicious use of these agents increases airflow and reduces dyspnea in patients with COPD. Patients often experience a reduction in symptoms and improvement in their quality of life. There are several classes of bronchodilators available for the treatment of COPD, each with specific clinical benefits: anticholinergics, short-acting beta 2 agonists, combination anticholinergic and short-acting beta 2 agonist, long-acting beta 2 agonists and methylxanthines. Anticholinergics such as ipratropium bromide have been used concomitantly with other bronchodilators for the treatment of patients with COPD. Ipratropium bromide is a quaternary anticholinergic bronchodilator that is commonly used to treat obstructive lung disease. Although ipratropium is not usually employed as a first-line bronchodilator to treat chronic asthma, it has been used extensively in hospital emergency departments as adjunctive therapy for the emergency treatment of acute asthma exacerbation.

ARDS' most common symptoms are labored, rapid breathing, nasal flaring, cyanosis blue skin, lips and nails caused by lack of oxygen to the tissues, breathing difficulty, anxiety, stress, tension, joint stiffness, pain and temporarily absent breathing. In the following paragraphs, the specific conditions will be described, and the existing treatments, if any, discussed. ARDS is currently diagnosed by mere symptomatic signs, e.g. chest auscultation with a stethoscope that may reveal abnormal symptomatic breath sounds, and confirmed with chest X-rays and the measurement of arterial blood gas. ARDS, in some instances, appears to be associated with other diseases, such as acute myelogenous leukemia, acute tumor lysis syndrome (ATLS) developed after treatment with, e.g. cytosine arabinoside, etc. In general, however, ARDS is associated with traumatic injury, severe blood infections such as sepsis or other systemic illness, high-dose radiation therapy and chemotherapy, and inflammatory responses which lead to multiple organ failure and in many cases death. In premature babies (“premies”), the lungs are not quite developed and, therefore, the fetus is in an anoxic state during development. Moreover, lung surfactant, a material critical for normal respiration, is generally not yet present in sufficient amounts at this early stage of life; however, premies often hyper-express the adenosine A1 receptor and/or underexpress the adenosine Ata receptor and are, therefore, susceptible to respiratory problems including bronchoconstriction, lung inflammation and ARDS, among others. When Respiratory Distress Syndrome (RDS) occurs in premies, it is an extremely serious problem. Preterm infants exhibiting RDS are currently treated by ventilation and administration of oxygen and surfactant preparations. When premies survive RDS, they frequently develop bronchopulmonary dysplasia (BPD), also called chronic lung disease of early infancy, which is often fatal.

Rhinitis may be seasonal or perennial, allergic or non-allergic. Non-allergic rhinitis may be induced by infections, such as viruses, or associated with nasal polyps, as occurs in patients with aspirin idiosyncrasy. Medical conditions such as pregnancy or hypothyroidism and exposure to occupational factors or medications may cause rhinitis. The so-called NARES syndrome is a non-allergic type of rhinitis associated with eosinophils in the nasal secretions, which typically occurs in middle-age and is accompanied by some loss of sense of smell. When cholinergic pathways are stimulated they produce typical secretions that are identified by their glandular constituents so as to implicate neurologic stimulation. Other secretions typical of increased vascular permeability are found in allergic reactions as well as upper respiratory infections, and the degranulation of mast cells releases preformed mediators that interact with various cells, blood vessels, and mucous glands, to produce the typical rhinitis symptoms. Most early- and late-phase reactions occur in the nose after allergen exposure. The late-phase reaction is seen in chronic allergic rhinitis, with hypersecretion and congestion as the most prominent symptoms. When priming occurs, it exhibits a lowered threshold to stimulus after repeated allergen exposure which, in turn, causes a hypersensitivity reaction to one or more allergens. Sufferers may also become hyper-reactive to non-specific triggers such as cold air or strong odors. Self-administered saline improves nasal stuffiness, sneezing, and congestion and usually causes no side effects and it is, thus, the first treatment tried in pregnant patients. Saline sprays are generally used to relieve mucosal irritation or dryness associated with various nasal conditions, minimize mucosal atrophy, and dislodge encrusted or thickened mucus. If used immediately before intranasal corticosteroid dosing, saline sprays may help prevent drug-induced local irritation. Anti-histamines such as terfenadine and astemizole, two non-sedating anti-histamines, are also employed to treat this condition, but have been associated with a ventricular arrhythmia known as Torsades de Points, usually in interaction with other medications such as ketoconazole and erythromycin, or secondary to an underlying cardiac problem. Loretadine, another non-sedating anti-histamine, and cetirizine have not been associated with an adverse impact on the QT interval, or with serious adverse cardiovascular events. Cetirizine, however, produces extreme drowsiness and has not been widely prescribed. Non-sedating anti-histamines, e.g. Claritin, may produce some relieving of sneezing, runny nose, and nasal, ocular and palatal itching, but have not been tested for asthma or other more specific conditions. Terfenadine, loratadine and astemizole, on the other hand, exhibit extremely modest bronchodilating effects, reduction of bronchial hyper-reactivity to histamine, and protection against exercise- and antigen-induced brorichospasm. Some of these benefits, however, require higher-than-currently-recommended doses. The sedating-type anti-histamines help induce night sleep, but they cause sleepiness and compromise performance if taken during the day. When employed, anti-histamines are typically combined with a decongestant to help relieve nasal congestion. Sympathomimetic medications are used as vasoconstrictors and decongestants. The three commonly prescribed systemic decongestants, pseudoephedrine, phenylpropanolamine and phenylephrine cause hypertension, palpitations, tachycardia, restlessness, insomnia and headache. The interaction of phenylpropanolamine with caffeine, in doses of two to three cups of coffee, may significantly raise blood pressure. In addition, medications such as pseudoephedrine may cause hyperactivity in children. Topical decongestants, nevertheless, are only indicated for a limited period of time, as they are associated with a rebound nasal dilatation with overuse.

Anti-cholinergic agents are given to patients with significant rhinorrhea or for specific conditions such as “gustatory rhinitis”, usually caused by ingestion of spicy foods, and may have some beneficial effects on the common cold. Cromolyn, for example, if used prophylactically as a nasal spray, reduces sneezing, rhinorrhea, and nasal pruritus, and blocks early- and late-phase hypersensitivity responses, but produces sneezing, transient headache, and even nasal burning. Topical corticosteroids such as Vancenase are somewhat effective in the treatment of rhinitis, especially for symptoms of congestion, sneezing, and runny nose. Depending on the preparation, however, corticosteroid nose sprays may cause irritation, stinging, burning, or sneezing, as well. Local bleeding and septal perforation can also occur sometimes, especially if the aerosol is not aimed properly. Topical steroids generally are more effective than cromolyn sodium, particularly in the treatment of NARES, and also to reduce some symptoms of rhinitis. Their side effects, however, limit their usefulness except for temporary therapy in patients with severe symptoms. These agents are sometimes used for shrinking nasal polyps when local therapy fails. Immunotherapy, while expensive and inconvenient, often provides benefits, especially for inpatients who experience-side effects from other medications. So-called blocking antibodies, and agents that alter cellular histamine release, eventually result in decreased IgE, along with many other favorable physiologic changes. This effect is useful in IgE-mediated diseases, e.g., hypersensitivity in atopic patients with recurrent middle ear infections. For allergic rhinitis sufferers, however, a runny nose is more than a nuisance. The disorder often results in impaired quality of life and sets the stage for more serious ailments, including psychological problems. Presently, rhinitis is mostly treated with propranolol, verapamil, and adenosine, all of which have Food and Drug Administration-approved labeling for acute termination of supraventricular tachycardia (SVT). The non-glucocorticoid steroids of this invention are believed to be substantially free of the listed detrimental effects of those steroids currently in use.

Although the progress and symptoms of pulmonary fibrosis and other interstitial lung diseases (ILD) may vary, they all affect parts of the lung. When inflammation involves the walls of the bronchioles (small airways) it is called bronchiolitis, when it involves the walls and air spaces of the alveoli (air sacs) it is called alveolitis, and when it involves the small blood vessels (capillaries) of the lungs it is called vasculitis. The inflammation may heal, or it may lead to permanent scarring of the lung tissue, in which case it is called pulmonary fibrosis. This fibrosis or scarring of the lung tissue results in permanent loss of its ability to breathe and carry oxygen, and the amount of scarring determines the level of disability a person experiences because of the destruction by the scar tissue of the air sacs and lung tissue between and surrounding the air sacs and the lung capillaries. When this happens, oxygen is generally administered to help improve breathing. Pulmonary fibrosis is caused by, or takes the form of, occupational and environmental exposure to irritants such as asbestos, silica and metal dusts, bacteria and animal dusts, gases and fumes, asbestosis and silicosis, infections that produce lung scarring, of which tuberculosis is one example, connective tissue or collagen diseases such as Rheumatoid Arthritis, Systemic Sclerosis and Systemic Lupus Erythematosis, idiopathic pulmonary fibrosis and, although not as common, pulmonary fibrosis of genetic/familial origin and certain medicines. Many of the diseases are often named after the occupations with which they are associated, such as Grain handler's lung, Mushroom worker's lung, Bagassosis, Detergent worker's lung, Maple bark stripper's lung, Malt worker's lung, Paprika splitter's lung, and Bird breeder's lung. “Idiopathic” (of unknown origin) pulmonary fibrosis (IPF) is the label applied when all other causes of interstitial lung disease have been ruled out, and is said to be caused by viral illness and allergic or environmental exposure (including tobacco smoke). Bacteria and other microorganisms are not thought to be a cause of IPF. There is also a familial form of the disease, known as familial idiopathic pulmonary fibrosis, whose main symptom is shortness of breath, which is difficult to diagnose since many lung diseases show this symptom. The shortness of breath may first appear during exercise, eventually resulting in shortness of breath even at rest. Other symptoms may include dry cough (without sputum), and clubbing of the fingertips. Glucocorticosteroids are usually administered to treat pulmonary fibrosis inflammation with inconclusive results. Other drugs, however, are not usually added until it is clear that the steroids are not effective in reversing the disease. Glucocorticosteroids are also used in combination with other drugs when diagnosis is first established, e.g. oxygen therapy that is prescribed in severe cases. The administration of influenza and pneumococcal pneumonia vaccines is often recommended in pulmonary fibrosis and more generally for all lung diseases to prevent infection. The treatment and management of pulmonary fibrosis often requires a lung biopsy to assess the unpredictable response of patients to glucocorticosteroids or other immune system suppressants. Lung transplants are sometimes an ultimate option in severe cases of pulmonary fibrosis and other lung diseases.

Pulmonary fibrosis may also be caused by other specific diseases, such as sarcoidosis, a disease whose cause is unknown, that is characterized by the formation of granulomas or areas of inflammatory cells. This disease may attack any organ of the body, but most frequently attacks the lungs, and is generally diagnosed when a chest X-ray shows enlarged lymph glands in the center of both lungs or evidence of lung tissue thickening. For many, sarcoidosis is a minor problem, and symptoms including dry cough, shortness of breath, mild chest pain, fatigue, weakness and weight loss—may appear infrequently and stop even without medication. For others, it is a disabling disease. Histiocytosis X, also associated with pulmonary fibrosis, seems to begin in the bronchioles or small airways of the lungs and their associated arteries and veins, and is generally followed by destruction of the bronchioles and narrowing and damaging of small blood vessels. Symptoms of this disease include dry cough (without sputum), breathlessness upon exertion, and chest pain, and may be chronic with loss of lung function. Glucocorticosteroid therapy is often prescribed, although there is no evidence that it is effective. Histiocytosis X is associated with cigarette smoking, and with jobs, such as mining and may be caused by inhalation of small particulate matter, e.g. dust or asbestos fibers that damage the lungs, especially the small airways and air sacs, and cause scarring (fibrosis). Agricultural workers are also affected by some particulate organic substances, such as moldy hay, which cause an allergic reaction in the lung called “Farmer's Lung”, and may cause pulmonary fibrosis as well. Asbestosis and silicosis are two occupational lung diseases whose causes are known. Asbestosis is caused by small needle-like particles of asbestos inhaled into the lungs, and cause lung scarring or pulmonary fibrosis that may lead to lung cancer. Silicosis is a dust disease that comes from breathing in free crystalline silica dust, and is produced by all types of mining in which the ore, e.g. gold, lead, zinc, copper, iron, anthracite (hard) coal, and some bituminous (soft) coal, are extracted from quartz rock. Workers in foundries, sandstone grinding, tunneling, sandblasting, concrete breaking, granite carving, and china manufacturing also encounter silica. Large silica particles are stopped in the upper airways, but the tiniest specks of silica are carried down to the lung alveoli, where they lead to pulmonary fibrosis. The use of glucocorticosteroids alone, or combined drug therapy, and the hope of lung transplant are three treatment approaches that are currently being tested, but up to the present time there is no good therapy for this disease. This patent provides the first effective therapy for these and other respiratory and lung ailments.

Neutrophilic inflammation is gaining increasing recognition as an important component of chronic persistent asthma, and is frequently associated with cases of fatal asthma. Neutrophilic inflammation is also considered to be the principal component of COPD. None of the currently available steroids are capable of attenuating neutrophilic inflammatory reactions, and may in fact contribute to them by delaying neutrophil apoptosis. DHEA-S has been shown to be effective in three different models of human asthma: the allergic rabbit, the allergic mouse, and the allergic primate. These effects included decreased magnitude of both early phase and late stage responses following allergen challenge, and dramatic reduction in eosinophilic and neutrophilic inflammation. DHEA-S was found to be at least equivalent to budesonide, with respect to reduction of eosinophilic inflammation, and far superior to it with respect to neutrophilic inflammation. With respect to reduction of eosinophilic inflammation, DHEA-S was found to be at least equivalent to budesonide (Pulmicort) and far superior to it with respect to neutrophilic inflammation. Very few putative asthma therapeutics show evidence of activity in multiple animal models. DHEA and DHEA-S are naturally occurring steroids found in all tissues of the body of both males and females. Low dose inhalation therapy with the steroids of this invention is therefore well tolerated. In fact, there is extensive information on the safe use of DHEA-S in humans, including in pregnant females, albeit by other routes of administration, such as orally. The present steroidal compounds are believed to work by a mechanism of action different from glucocorticoid steroids; that is these compounds do not appear to activate the glucocorticoid receptor. One of its demonstrated effects is the reduction of pulmonary adenosine, a potentially critical feature in view of the role of adenosine in pulmonary inflammation, and the fact that the lungs of asthma contain excessive amounts of this autocoid. Because the present steroids function by a different mechanism, they are not expected to exhibit any of the classical side effects of glucocorticoid steroids, e.g., mucositis, skin thickening, exopthalmia, reductions in bone growth (children) or/and mineralization (adults). Preclinical toxicology studies show that even at doses as high as 2 mg/kg/day in dogs and 11 mg/kg/day in rats, a maximum tolerated dose is not achieved. These doses are substantially in excess of the clinical dose in either asthma or COPD patients.

The use of the present steroids in the treatment of respiratory diseases is described in U.S. Pat. No. 6,087,351 to Nyce. That patent relates to the use of inhaled formulations of the steroids for treating diseases associated with altered adenosine levels, e.g., asthma. No commercially available steroid is capable of addressing the neutrophilic inflammation central to both asthmatic and COPD disease processes. The present steroids are the first capable of simultaneously inhibiting both eosinophilic and neutrophilic inflammation. To be administered to a subject are a first active agent selected from dehydroepiandrosterone, analogues or their pharmaceutically or veterinarily acceptable salts, and a second active agent selected from anti-muscarinic agents, alone or in conjunction with anti-histamines, anti-sense oligonucleotides, leukotriene inhibitors, theophyllines, β2 adrenergic agents, and/or mucolytics, among others. The first and second agents are administered in therapeutic or prophylactic amounts that are effective to inhibit, delay or control symptoms of the treated diseases or conditions, particularly those associated with lung vasoconstriction, bronchoconstriction, lung inflammation, lung allergies, changes in lung tissues, immune cell accumulation, e.g., neutrophils and eosinophils, fibrosis, cancerous tissue development, and others. More specifically, in one embodiment the pharmaceutical or veterinary composition of the invention comprises a first active agent selected from a non-glucocorticoid steroid having the chemical formula I shown below:

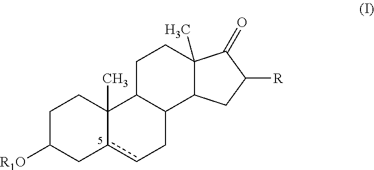

wherein the broken line represents a single or a double bond; R is hydrogen or a halogen; the H at position 5 is present in the alpha or beta configuration or the compound of chemical formula I comprises a racemic mixture of both configurations; and R¹ is hydrogen or SO₂OM, wherein M is selected from the group consisting of H Na, sulfatide

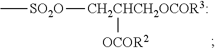

and phosphatide:

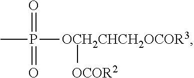

wherein R¹ and R³, which may be the same or different, are straight or branched (C₁-C₁₄) alkyl or glucuronide:

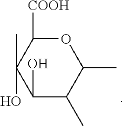

In the compound of formula (I), R preferably is halogen e.g., bromo, chloro, or fluora, R₁ is H, and the double bond is present, more preferably the compound of formula (I) is 16-alpha-fluoro epiandrosterone in a preferred embodiment of the compound of formula (I), R is H, R₁ is SO₂OM, M is a sulphatide group and the double bond is present, and more preferably the compound of formula (I) is the dehydrated form of dehydroepiandrosterone sodium sulphate (DHEA-S2H₂O) of chemical formula (II) shown below:

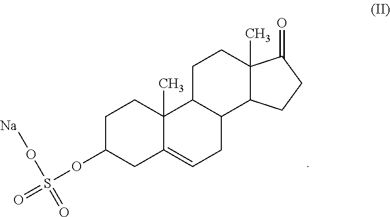

In still another embodiment, the non-glucocorticoid steroid has the chemical formula III, and IV shown below:

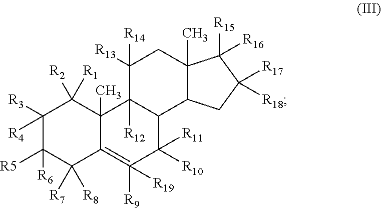

non-glucocorticoid steroid of the chemical formula:

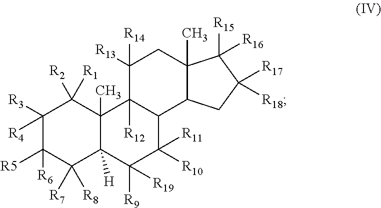

wherein R1, R2, R3, R4, R5, R7, R8, R9, R10, R12, R13, R14 and R19 are independently H, OR, halogen, (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, -OSO2R20, -OPOR20R21 or (C1-C10) alkyl, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H, halogen, (C1-C10) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, -OSO2R20 or -OPOR2OR21 when R16 is H, or R15 and R16 taken together are ═O; R17 and R18 are independently (1) H, —OH, halogen, (C1-C10) alkyl or —(C1-C10) alkoxy when R6 is H OR, halogen. (C1-C10) alkyl or —C(O)OR22, (2) H, (C1-C10 alkyl)amino, ((C1-C10) alkyl)n amino-(C1-C10) alkyl, (C1-C10) alkoxy, hydroxy-(C1-C10) alkyl, (C1-C10) alkoxy —(C1-C10) alkyl, (halogen)m (C1-C10) alkyl, (C1-C10) alkanoyl, formyl, (C1-C10) carbalkoxy or (C1-C10) alkanoyloxy when R15 and R16 taken together are ═O, (3) R17 and R18 taken together are ═O; (4) R17 or R18 taken together with the carbon to which they are attached form a 3-6 member ring containing 0 or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)m (C1-C10) alkyl or (C1-C10) alkyl; n is 0, 1 or 2; and m is 1, 2 or 3; or pharmaceutically or veterinarily acceptable salts thereof.

In still another embodiment, the steroid may be a compound of chemical formula V:

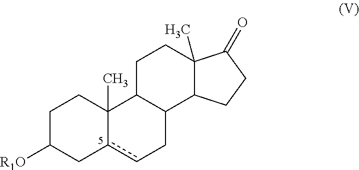

or pharmaceutically or veterinarily acceptable slats thereof; wherein R1 is A—CH(OH)—C(O)—, and A is H or (C1-C22) alkyl, alkenyl, or alkynyl, each of which may be substituted with one or more (C1-C4) alkyl, halogen, HO, or phenyl which may be substituted with, one or more halogen, HO, CH₃, or CH₃O.

The hydrogen atom at position 5 of the chemical formula I may be present in the alpha or beta configuration, or the DHEA compound may be provided as a mixture of compounds of both configurations. Compounds illustrative of chemical formula I above are included, although not exclusively, are DHEA, wherein R and R¹ are each hydrogen, containing a double bond; 16-alpha bromoepiandrosterone, wherein R is Br, R¹ is H, containing a double bond; 16-alpha-fluoro epiandrosterone, wherein R is F, R¹ is H, containing a double bond; Etiocholanolone, wherein R and R¹ are each hydrogen lacking a double bond; and Dehydroepiandrosterone sulphate, wherein R is H, R¹ is SO₂OM and M is a sulphatide group as defined above, lacking a double bond. Others, however, are also included. Also preferred compounds of formula I are those where R is halogen, e.g. bromo, chloro, or fluoro, where R1 is hydrogen, and where the double bond is present. A most preferred compound of formula I is 16-alpha-fluoro epiandrosterone. Other preferred compounds are DHEA and DHEA salts, such as the sulfate salt (DHEA-S). One most preferred composition and treatment involves DHEA-S and Ipratropium Bromide, and another involves DHEA-S and Tiotropium Bromide. It is believed that the non-glucocorticoid steroid and the anti-muscarinic agents potentiate each other's anti-inflammatory effects whether administered orally, topically or through the respiratory tract. Other preferred combinations involves analogues of DHEA shown in the chemical formulas provided above and one or more of the anti-muscarinic agents. Other DHEA analogues and derivatives suitable for use in this invention are non-glucocorticoid steroid of the chemical formula

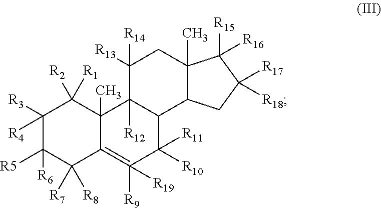

or a non-glucocorticoid steroid of the chemical formula

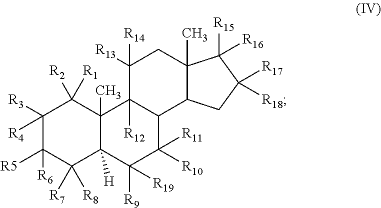

wherein R1, R2, R3, R4, R5, R7, R8, R9, R10, R12, R13, R14 and R19 are independently H, OR, halogen, (C1-C10) alkyl or (C1-C10) alkoxy, R5 and R11 are independently OH, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, -OSO2R20, -OPOR2OR21 or (C1-C10) alkyl, R5 and R6 taken together are ═O, R10 and R11 taken together are ═O; R15 is (1) H, halogen, (C1-C10) alkyl, or (C1-C10) alkoxy when R16 is —C(O)OR22, (2) H, halogen, OH or (C1-C10) alkyl when R16 is halogen, OH or (C1-C10) alkyl, (3) H, halogen, (C1-C1O) alkyl, (C1-C10) alkenyl, (C1-C10) alkynyl, formyl, (C1-C10) alkanoyl or epoxy when R16 is OH, (4) OR, SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, -OSO2R20 or -OPOR20R21 when R16 is H, or R15 and R16 taken together are ═O; R17 and R18 are independently (1) H, —OH, halogen, (C1-C10) alkyl or —(C1-C10) alkoxy when R6 is H OR, halogen (C1-C10) alkyl or —C(O)OR22, (2) H, (C1-CtO alkyl)amino, 9(C1-C10) alkyl)n amino-(C1-C1O) alkyl, (C1-CtO) alkoxy, hydroxy-(C1-C10) alkyl, (C1-C10) alkoxy-(C1-C10) alkyl, (halogen)m (C1-C10) alkyl, (C1-C10) alkanoyl, formyl, (C1-C10) carbalkoxy or (C1-C10) alkanoyloxy when R15 and R16 taken together are ═O, (3) R17 and B.18 taken together are ═O; (4) R17 or R18 taken together with the carbon to which they are attached form a 3-6 member ring containing 0 or 1 oxygen atom; or (5) R15 and R17 taken together with the carbons to which they are attached form an epoxide ring; R20 and R21 are independently OH, pharmaceutically acceptable ester or pharmaceutically acceptable ether; R22 is H, (halogen)m (C1-C10) alkyl or (C1-C10) alkyl; n is 0, 1 or 2; and m is 1, 2 or 3; or pharmaceutically or veterinarily acceptable salts thereof. Of the non-glucocorticoid steroids of formulas (III) and (IV), preferred are those where R15 and R16 together are ═O, also preferred are those where R5 is OH, where R5 is -OSO2R20, and where R20 is H.

In general, the non-glucocorticoid steroids, such as those of formulas (I), (II), (III), (IV) and (V), their derivatives and their salts are administered in a dosage of about 0.05, about 0.1, about 1, about 5, about 20 to about 100, about 500, about 1000, about 1500 about 1,800, about 2500, about 3000, about 3600 mg/kg body weight. Other dosages, however, are also suitable and are contemplated within this patent. The first active agent of formula I, III and IV may be made in accordance with known procedures, or variations thereof that will be apparent to those skilled in the art. See, for example, U.S. Pat. No. 4,956,355; UK Patent No. 2,240,472; EPO Patent Application No. 429; 187, PCT Patent Publication No. WO 91/04030; U.S. Pat. No. 5,859,000; Abou-Gharbia et al., J. Pharm. Sci. 70: 1154-1157 (1981); Merck Index Monograph No. 7710 (11th Ed. 1989), among others. The dehydroepiandrosterone and its salts may be administered with the second agent for e.g. ipratropium bromide, and optionally a non-glucocorticoid steroid of formula (I), (II), (III), (IV) or (V), and/or other bioactive agents, separately and concurrently, before or after one another, or in the same composition. In many cases, the dosage lends itself to simultaneous administration, either in the same composition or separately for example once a day. Among the other bioactive agents, preferred is the administration of any of the currently prescribed drugs for asthma, COPD, allergic rhinitis, etc. These include β-2 adrenergic agonists such as ephedrine, isoproterenol, isoetharine, epinephrine, metaproterenol, terbutaline, fenoterol, procaterol, albuterol, salbutamol, pirbuterol, formoterol, bilateral, bambuterol, salmeterol and seretide, among others; other anti-cholinergic agents; anti-histaminic agents; adenosine A1, A2b and A3 receptor antagonists such as anti-sense oligos, among others; adenosine Ata agonists; and glucocorticosteroids. The phrase “concurrently administering”, as used herein, means that the anti-inflammatory steroid or its salt and the anti-muscarinic agents are administered either simultaneously in time (preferably by formulating the two together in a common pharmaceutical carrier), or at different times during the course of a common treatment schedule. In the case where both DHEA and anti-muscarinic agents are administered, they may be administered at times sufficiently close or simultaneously to enhance their anti-inflammatory effects. The anti-muscarinic agent and the non-glucocorticoid steroid or their salts may be formulated individually with a pharmaceutically acceptable carrier, or with the second active agent. The non-glucocorticoid steroid, and its salts and the anti-muscarinic agent may be administered systematically, topically, or directly into the respiratory tract of the subject. The composition may be formulated by any of the techniques set forth in this patent and others as an artisan would know.

In general, the anti-muscarinic agent(s) is (are) administered in a therapeutic amount for treating the targeted disease or condition, and/or an amount effective to reduce or inhibit undesirable symptoms in the lungs of the subject, and the dosage will vary depending upon the condition of the subject, other agents being administered, the type of formulation employed, and the route of administration. Generally, the anti-muscarinic agents may be administered in amounts known in the art. However, these amounts may be reduced, as an artidan will know for joint administration with the dehydroxyepiandrosterone(s) of the invention. Other amounts may, of course, be employed as well in accordance with the state of the patient, other agents administered and route of administration, as an artisan would know. The anti-muscarinic receptor agent may be administered once, or several times, a day. The non-glucocorticoid steroid, anti-muscarinic agents, and any other optional drugs, anti-sense oligos to adenosine receptors or other targets, used to treat respiratory, lung and neoplastic diseases, and other agents listed below, may be administered per se or in the form of pharmaceutically acceptable salts, as discussed above, all being referred to as “active compounds or agents.” The active compounds or their salts may be administered either systemically or topically, as discussed below.

Examples of anti-muscarinic agents are ipratropium bromide, fiotropium bromide, troventol, and others known in the art. A recent publication by Yahgmurov reported improvement in pulmonary function with enhancement of airway conductance in large and middle bronchi in COPD patients after treatment with troventol. In bronchial asthma (BA) patients, a lesser effect on airway resistance was observed, which appeared more noticeable in small bronchi. The administration of troventrol by inhalation to patients afflicted with chronic obstructive pulmonary disease (COPD) was reported in the same publication to result in a reduction in the generation of reactive oxygen species by phagocytes in blood and bronchioalveolar lavage (BAL). The absence of inhibition of peroxide lipid oxidation (PLO) is noted, and explained as due to a residual phenomenon of “respiratory burst”. Monotherapy with troventol in BA patients, unlike in COPD patients, was reported to lead to PLO reduction only in BAL. The level of serum calcium is said to have changed in a non-significant fashion after treatment. See, B. H. Yahgmurov, Pulmonology Journal, Russian Pneumologycal Scientific Society, Ministry Public Health of Russian Federation Vol. 6 (4) (1996). In addition, the same author assessed the effect of troventol on histamine release from mast cell, one step of the initial chain in bronchospasm. When troventol was compared with atrovent (ipratropium bromide) and atropine, the author reports that cell incubation with troventol for 5 min inhibited histamine release by 47%, and no distinction was observed between placebo and atropine or atrovent effects. The histamine release from mast cells is explained as having been caused by an abrupt increase in the concentration of cytosolic calcium ion. The speed of passive calcium ion uptake was decreased by troventol (56.3%) and by atrovent (28%), but not by atropine. Thus, troventol, unlike atrovent and atropine, appears to inhibit histamine secretion by reducing the cell membrane permeability of calcium ions. Examples of bronchodilating agents other than anti-muscarinic agents are ubiquinones, glucocorticoids, adenosine receptor antagonists such as theophyllines, anti-cholinergics, and β2 adrenergic agonists. Examples of leukotrienes are zyflo, an inhibitor of the enzyme 5-lipoxygenase (5-LO), zafirlukast (Accolate®), montelukast (Singulair®), and others known in the art. In six-month clinical trials involving patients with mild-to-moderate asthma who used daily-inhaled beta-agonists, Zyflo improved lung function, and decreased % patients requiring steroid rescue for worsening asthma compared to patients treated with placebo. Overall, the patients receiving Zyflo also requiring steroid rescue was only 7%, compared with 18.7% placebo, a reduction of 62%. Patients receiving Zyflo also were able to reduce their use of inhaled beta-agonists, and at the end of the study the number of beta-agonist puffs needed per day was 1.77 puffs, or 31% lower than baseline in Zyflo-treated patients, with a 0.22 puff decrease in the placebo group. Leukotriene receptor antagonists (LTRAs) inhibit the effects of the cysteinyl leukotrienes, which represent 3 of a large number of chemical mediators of asthma. Leukotrienes are released by several types of cells and can cause bronchoconstriction and inflammation. The cysteinyl leukotrienes are particularly important mediators in patients with aspirin-sensitive asthma (characterized by chronic severe asthma symptoms, nasal polyps; and aspirin-induced bronchospasm). LTRAs competitively block leukotriene receptors on bronchial smooth muscle and elsewhere. Examples of β2 adrenergic agonists are ephedrine, isoproterenol, isoetharine, epinephrine, metaproterenol, terbutaline, fenoterol, procaterol, albuterol, salmeterol, pirbuterol, formoterol, biloterol, bambuterol, salbutamol, and seretide, among others. Examples of glucocorticosteroids, such as beclomethasone, corticoid 21-sulfopropionates, (16 alpha)-16,17-alkylidene bis(oxy)-3-arylpregna-2,4-trien-20-ones, hydrocortisone esters, cyproterone thiopivalate (CTP), hydrocortisone, dexamethasone trimethyl acetate, alkane sulfonic acids of decinine, α-hydroxyprednisolone, 18,18-difluorosteroids, preparing 17.alpha-hydroxy corticoid 21-phosphate, 21-phosphate corticoids having unprotected hydroxyl radicals at least at the 17.alpha- and 21-position, 16.alpha.-methylated δ-17(20)-corticoids, 21-(L-ascorbyl-2-phosphoryl) dexamethasone, 21-(L ascorbyl-2-phosphoryl)hydrocortisone, 21-(L-ascorbyl-2-phosphoryl) triamcinolone acetonide and physiologically acceptable salts thereof, among others. Some of these are effective for short periods of time, but in conjunction with the non-glucocorticoid steroids provide a good combination of short and long term relief.

The daily dosage of the anti-muscarinic agent and the non-glucocorticoid steroid to be administered to a subject will vary with the overall treatment programmed, the agent employed, the type of formulation, the route of administration and the state of the patient. Anti-muscarinic agents and anti-inflammatory steroids are known in the art, and are commercially available. Examples 16 to 26 show aerosolized preparations in accordance with the invention for delivery with a device for respiratory or nasal administration, or administration by inhalation. For intrapulmonary administration, liquid preparations are preferred. In the case of other bioactive agents, there exist FDA recommended amounts for supplementing a person's dietary intake with additional bioactive agents, such as in the case of vitamins and minerals. However, where employed for the treatment of specific conditions or for improving the immune response of a subject they may be utilized in dosages hundreds and thousands of times higher. Mostly, the pharmacopeia's recommendations cover a very broad range of dosages, from which the medical artisan may draw guidance. Amounts for the exemplary agents described in this patent may be in the range of those currently being recommended for daily consumption, below or above those levels. The treatment may typically begin with a low dose of an anti-muscarinic agent in combination with a non-glucocorticoid steroid, and optionally a glucocorticoid steroid or other bioactive agent as appropriate, and then a titration up of the dosage for each patient. Higher and smaller amounts, including initial amounts, however, may be administered within the confines of this invention as well. The dosage of each of the agents should be adjusted downwards to begin therapy until a dosage is reached that is adequate for the patient. It is recommended that, when possible, a once-a-day dose be administered to maintain a continuous blood level of the agent. Preferable ranges for the first, second and other agents employed here will vary depending on the route of administration and type of formulation employed, as an artisan will appreciate and manufacture in accordance with known procedures and components. The active compounds may be administered as one dose (once a day) or in several doses (several times a day). The compositions and method of preventing and treating respiratory and neoplastic diseases may be used to treat adults, children and infants, as well as non-human animals afflicted with the described conditions. Although the present invention is concerned primarily with the treatment of human subjects, it may also be employed, for veterinary purposes in the treatment of other mammalian subjects, such as dogs and cats as well as for large domestic and wild animals. Thus, this treatment helps regulate (titrate) the patient in a custom tailored manner. Whereas the administration of an agent such as the non-glucocorticoid steroid in accordance with this invention may reduce inflammation and bronchoconstriction, the further administration of an anti-muscarinic agent will improve the subject's respiration in a short period of time.

Other agents that may be incorporated into the present composition or administered in conjunction with this therapy are one or more of a variety of therapeutic agents that are administered to humans and animals. Some of the categories of agents suitable are analgesics, pre-menstrual medications, menopausal agents, anti-aging agents, anti-anxiolytic agents, mood disorder agents, anti-depressants, anti-bipolar mood agents, anti-schizophrenic agents, anti-cancer agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, analgesics, pre-menstrual medications, anti-menopausal agents such as hormones and the like, anti-aging agents, anti-anxiolytic agents, nociceptic agents, mood disorder agents, anti-depressants, anti-bipolarmood agents, anti-schizophrenic agents, anti-cancer agents, alkaloids, blood pressure controlling agents, hormones, anti-inflammatory agents, other agents suitable for the treatment and prophylaxis of diseases and conditions associated or accompanied with pain and inflammation, such as arthritis, burns, wounds, chronic bronchitis, chronic obstructive pulmonary disease (COPD), inflammatory bowel disease such as Crohn's disease and ulcerative colitis, autoimmune disease such as lupus erythematosus, muscle relaxants, steroids, soporific agents, anti-ischemic agents, anti-arrhythmic agents, contraceptives, vitamins, minerals, tranquilizers, neurotransmitter regulating agents, wound and burn healing agents, anti-angiogenic agents, cytokines, growth factors, anti-metastatic agents, antacids, anti-histaminic agents, antibacterial agents, anti-viral agents, anti-gas agents, agents for reperfusion injury, counteracting appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, etc.

Among the hormones are female and male sex hormones such as premarin, progesterone, androsterones and their analogues, thyroxine and glucocorticoids, among the libido altering agents are Viagra and other NO-level modulating agents, among the analgesics are over-the-counter medications such as ibuprofen, oruda, aleve and acetaminophen and controlled substances such as morphine and codeine, among the anti-depressants are tricyclics, MAO inhibitors and epinephrine, γ-amino butyric acid (GABA), dopamine and serotonin level elevating agents, e.g. Prozac, Amytryptilin, Wellbutrin and Zoloft, among the skin renewal agents are Retin-A, hair growth agents such as Rogaine, among the anti-inflammatory agents are non-steroidal anti-inflammatory drugs (NSAIDs) and steroids, among the soporifics are melatonin and sleep inducing agents such as diazepam, cytoprotective, anti-ischemic and head injury agents such as enadoline, and many others. Examples of agents in the different groups are provided in the following list. Examples of analgesics are Acetominophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Examples of anti-anxiety agents include Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Examples of anti-anxiety agents associated with mental depression are Chlordiazepoxide, Amitriptyline, Loxapine Maprotiline and Perphenazine, among others. Examples of anti-inflammatory agents are non-rheumatic Aspirin, Choline Salicylate, Diclofenac, Diflunisel, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin. Examples of anti-inflammatories for ocular treatment are Diclofenac, Flurbiprofen, Indomethacin, Ketorolac, Rimexolone (generally for post-operative treatment). Examples of anti-inflammatories for non-infectious nasal applications are Beclomethaxone, Budesonide, Dexamethasone, Flunisolide, Triamcinolone, and the like. Examples of soporifics (anti-insomnia/sleep inducing agents) such as those utilized for treatment of insomnia, are Alprazolam, Bromazepam, Diazepam, Diphenhydramine, Doxyiamine, Estazolam, Flurazepam, Halazepam, Ketazolam, Lorazepam, Nitruzepam, Prazepam Quazepam, Temazepam, Triazolam, Zolpidem and Sopiclone, among others. Examples of sedatives are Diphenhydramine, Hydroxyzine, Methotrimeprazinc, Promethazine, Propofol, Melatonin, Trimeprazine, and the like. Examples of sedatives and agents used for treatment of petit mal and tremors, among other conditions, are Amitriptyline HCl, Chlordiazepoxide, Amobarbital, Secobarbital, Aprobarbital, Butabarbital, Ethchiorvynol, Glutethimide, L-Tryptophan, Mephobarbital, MethoHexital Na, Midazotam HCl, Oxazepam, Pentobarbital Na, Phenobarbital, Secobarbital Na, Thiamytal Na, and many others. Agents used in the treatment of head trauma (Brain Injury/Ischemia) include Enadoline HCl (e.g. for treatment of severe head injury, orphan status, Warner Lambert). Examples of cytoprotective agents and agents for the treatment of menopause and menopausal symptoms are Ergotamine, Belladonna Alkaloids and Phenobarbitals. Examples of agents for the treatment of menopausal vasomotor symptoms are Clonidine, Conjugated Estrogens and Medroxyprogesterone, Estradiol, Estradiol Cypionate, Estradiol Valerate, Estrogens, conjugated Estrogens, esterified Estrone, Estropipate and Ethinyl Estradiol. Examples of agents far treatment of symptoms of Pre Menstrual Syndrome (PMS) are Progesterone, Progestin, Gonadotrophic Releasing Hormone, oral contraceptives, Danazol, Luprolide Acetate and Vitamin B6. Examples of agents for the treatment of emotional/psychiatric treatments are Tricyclic Antidepressants including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications. Examples of anti-migraine agents are Imitrex and the like.

The active agents of this invention are provided within broad amounts of the composition. For example, the active agents may be contained in the composition in amounts of about 0.001%, about 1%, about 2%, about 5%, about 10%, about 20%, about 40%, about 90%, about 98%, about 99.999% of the composition. The amount of each active agent may be adjusted when, and if, additional agents with overlapping activities are included as discussed in this patent. The dosage of the active compounds, however, may vary depending on age, weight, and condition of the subject. Treatment may be initiated with a small dosage, e.g. less than the optimal dose, of the first active agent of the invention, be it a non-glucocorticoid steroid or an anti-muscarinic agent that is administered first, and optionally other bioactive agents described above. This may be similarly done with the second active agent, until a desirable level is attained. Or vice versa, for example in the case of multivitamins and/or minerals, the subject may be stabilized at a desired level of these products and then administered the first active compound. The dose may be increased until a desired and/or optimal effect under the circumstances is reached. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, the dose of one of the other or of both agents may be adjusted to attain a desirable effect without exceeding a dose range that avoids untoward side effects. Thus, for example, when other analgesic and anti-inflammatory agents are added to the composition, they may be added in amounts known in the art for their intended application or in doses somewhat lower that when administered by themselves. Pharmaceutically acceptable salts should be pharmacologically and pharmaceutically or veterinarily acceptable, and may be prepared as alkaline metal or alkaline earth salts, such as sodium, potassium or calcium salts. Organic salts and esters are also suitable for use with this invention. The active compounds are preferably administered to the subject as a pharmaceutical or veterinary composition, which includes systemic and topical formulations. Among these, preferred are formulations suitable for inhalation, or for respirable, buccal, oral, rectal, vaginal, nasal, intrapulmonary, ophthalmic, optical, intracavitary, intratraccheal, intraorgan, topical (including buccal, sub lingual, dermal and intraocular), parenteral (including subcutaneous, intradermal, intramuscular, intravenous and intraarticular) and transdermal administration, slow release, implantable, and enteric coated, among others. The compositions may conveniently be presented in single or multiple unit dosage forms as well as in bulk, and may be prepared by any of the methods which are well known in the art of pharmacy. The actual preparation and compounding of these different formulations is known in the art and need not be detailed here. The active compounds may be administered once or several times a day. The composition of the invention may also be provided in the form of a kit, whether already formulated or where the active agents are separately provided along with other ingredients, and instructions for its formulation and administration regime. The kit may also contain other agents, such as those described in this patent and, for example, when for parenteral administration, they may be provided with a carrier in a separate container, where the carrier may be sterile. The present composition may also be provided in lyophilized form, and in a separate container, which may be sterile, for addition of a liquid carrier prior to administration. See, e.g. U.S. Pat. No. 4,956,355; UK Patent No. 2,240,472; EPO Patent Application Serial No. 429,187; PCT Patent Publication WO 91/04030; Mortensen, S. A., et al., Int. J. Tiss. Reac. XII(3): 155-162 (1990); Greenberg, S. et al., J. Clin. Pharm. 30: 596-608 (1990); Folkers, K., et al., P. N. A. S. (USA) 87: 8931-8934 (1990), the relevant preparatory and compound portions of which are incorporated by reference above.

Formulations suitable for respiratory, nasal, intrapulmonary, and inhalation administration are preferred, as are topical, oral and parenteral formulations. All methods of preparation include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and a suitable carrier. In general, the compositions of the invention are prepared by uniformly and intimately admixing the active compound with a liquid or finely divided solid carrier, or both, and then, if necessary, shaping the resulting mixture. For example, a tablet may be prepared by compressing or molding a power or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispensing agent(s). Molded tablets may be made by molding, in a suitable machine, the powdered compound moistened with an inert liquid binder. A syrup may be made by adding the active compound to a concentrated aqueous solution of a sugar, for example sucrose to which may also be added any accessory ingredients). Such accessory ingredient(s) may include flavorings, suitable preservatives, an agent to retard crystallization of the sugar, and an agent to increase the solubility of any other ingredient, such as a polyhydric alcohol, for example glycerol or sorbitol. Compositions for oral administration may optionally include enteric coatings known in the art to prevent degradation of the compositions in the stomach and provide release of the drug in the small intestine. Compositions suitable for buccal or sub-lingual administration include lozenges comprising the active compound in a flavored base, usually sucrose and acacia or tragacanth and pastilles comprising the compound in an inert base such as gelation and glycerin or sucrose and acacia.

Compositions suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Extemporaneous injection solutions and suspensions may be prepared from sterile powders, granules and tablets of the kind previously described.

Nasal and instillable formulations comprise purified aqueous solutions of the active compound with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye. Optical formulations are generally prepared in viscous carriers, such as oils and the like, as is known in the art, so that they may be easily administered into the ear without spilling.

Compositions suitable for topical application to the skin preferably take the form of an ointment, cream, lotion, paste, gel, spray, aerosol, or oil. Carriers which may be used include Vaseline, lanolin, polyethylene glycols, alcohols, transdermal enhancers, and combinations of two or more thereof. Compositions suitable for transdermal administration may be presented as discrete patches adapted to remain in intimate contact with the epidermis of the recipient for a prolonged period of time. Compositions suitable for transdermal administration may also be delivered by iontophoresis. See, for example, Pharmaceutical Research 3:318 (1986), and typically take the form of an optionally buffered aqueous solution of the active compound. Topical formulations comprise the active compound dissolved or suspended in one or more media such as mineral oil, petroleum, polyhydroxy alcohols or other bases used for topical pharmaceutical formulations. Cosmetic formulations may be in the form of solid or liquid preparations, for spreading on a subject's skin, including skin base, pancake, suntan, self-tanning and sun blocking lotions and oils. These formulations may additionally contain other cosmetic ingredients as are known in the art. Examples of these formulations are lotions, creams, oils, and other ointments, e.g. suntan lotions containing sunscreens and other protective ingredients, facial make-up and cleansing formulations, shampoos, hair and skin conditioners, and many more known in the art and commercially available. The addition of other accessory ingredients, vide infra, may be desirable, for example, accessory ingredient(s) selected from diluents, buffers, flavoring, coloring and aromatizing agents, binders, disintegrants, surface active agents, thickeners, lubricants, emulsifiers, surfactants, emollients, preservatives (including anti-oxidants), and the like. Other ingredients may also be utilized as is known in the art.

The active compounds disclosed herein may be administered into the respiratory system either by inhalation, respiration, nasal administration or intrapulmonary instillation (into the lungs) of a subject by any suitable means, and are preferably administered by generating an aerosol or spray comprised of powdered or liquid nasal, intrapulmonary, respirable or inhalable particles. The respirable or inhalable particles comprising the active compound are inhaled by the subject, i.e., by inhalation or by nasal administration or by instillation into the respiratory tract or the lung itself. The formulation may comprise respirable or inhalable liquid or solid particles of the active compound that, in accordance with the present invention, include respirable or inhalable particles of a size sufficiently small to pass through the mouth and larynx upon inhalation and continue into the bronchi and alveoli of the lungs. In general, particles ranging from about 0.05, about 0.1, about 0.5, about 1, about 2 to about 4, about 6, about 8, about 10 microns in size. More particularly, about 0.5 to less than about 5 microns in size, are respirable or inhalable. Particles of non-respirable size which are included in an aerosol or spray tend to deposit in the throat and be swallowed. The quantity of non-respirable particles in the aerosol is, thus, preferably minimized. For nasal administration or intrapulmonary instillation, a particle size in the range of about 8, about 10, about 20, about 25 to about 35, about 50, about 100, about 150, about 250, about 500 μm is preferred to ensure retention in the nasal cavity or for instillation and direct deposition into the lung. Liquid formulations may be squirted into the respiratory tract (nose) and the lung, particularly when administered to newborns and infants.

Liquid pharmaceutical compositions of active compound for producing an aerosol may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through, a 400 mesh screen to break up or separate out large agglomerates. A solid particulate composition comprised of the active compound may optionally contain a dispersant that serves to facilitate the formation of an aerosol. A suitable dispersant is lactose, which may be blended with the active compound in any suitable ratio, e.g., a 1 to 1 ratio by weight. Aerosols of liquid particles comprising the active compound may be produced by any suitable means, such as with a nebulizer. See, e.g. U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w composition, but preferably less than 20% w/w carrier being typically water or a dilute aqueous alcoholic solution, preferably made isotonic with body fluids by the addition of, for example sodium chloride. Optional additives include preservatives if the composition is not prepared sterile, for example, methyl hydroxybenzoate, anti-oxidants, flavoring agents, volatile oils, buffering agents and surfactants. Aerosols of solid particles comprising the active compound may likewise be produced with any sold particulate medicament aerosol generator. Aerosol generators for administering solid particulate medicaments to a subject product particles which are respirable, as explained above, and generate a volume of aerosol containing a predetermined metered dose of a medicament at a rate suitable for human administration. Examples of such aerosol generators include metered dose inhalers and insufflators.

Having now generally described this invention, the same will be better understood by reference to certain specific examples, which are included herein for purposes of illustration only and are not intended to be limiting of the invention or any embodiment thereof, unless so specified.

### EXAMPLES

In the following examples, DHEA means dehydroepiandrosterone, s means seconds, mg means milligrams, kg means kilograms, kw means kilowatts, Mhz means megahertz, and nmol means nanomoles.

### Examples 1 and 2

### In Vivo Effects of Folinic Acid & DHEA on Adenosine Levels

Young adult male Fischer 344 rats (120 grams) were administered dehydroepiandrosterone (DHEA) (300 mg/kg) or methyltestosterone (40 mg/kg) in carboxymethylcellulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen with 10 seconds of death. Liver and lungs were removed en bloc and flash frozen with 30 seconds of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 1 below. Results are expressed as the mean±SEM, with κp<0.05 compared to control group, and ψp<0.05 compared to DHEA or methyltestosterone-treated groups.

TABLE 1
In vivo Effect of DHEA, δ-1-methyltestosterone &
Folinic Acid on Adenosine Levels in various Rat Tissues

| | Intracellular Adenosine (nmols/mg protein) | | | |
|---|---|---|---|---|
| Treatment | Heart | Liver | Lung | Brain |
| Control | 10.6 ± 0.6 | 14.5 ± 1.0 | 3.1 ± 0.2 | 0.5 ± 0.04 |
| | (n = 12) | (n = 12) | (n = 6) | (n = 12) |
| DHEA | 6.7 ± 0.5 | 16.4 ± 1.4 | 2.3 ± 0.3 | 0.19 ± 0.01 |
| (300 mg/kg) | (n = 12) | (n = 12) | (n = 6) | (n = 12) |
| Methyltestosterone | 8.3 ± 1.0 | 16.5 ± 0.9 | N.D. | 0.42 ± 0.06 |
| (40 mg/kg) | (n = 6) | (n = 6) | | (n = 6) |
| Methyltestosterone | 6.0 ± 0.4 | 5.1 ± 0.5 | N.D. | 0.32 ± 0.03 |
| (120 mg/kg) | (n = 6) | (n = 6) | | (n = 6) |
| Folinic Acid | 12.4 ± 2.1 | 16.4 ± 2.4 | N.D. | 0.72 ± 0.09 |
| (50 mg/kg) | (n = 5) | (n = 5) | | (n = 5) |
| DHEA | 11.1 ± 0.6 | 18.8 ± 1.5 | N.D. | 0.55 ± 0.09 |
| (300 mg/kg) + | (n = 5) | (n = 5) | | (n = 5) |
| Folinic Acid | | | | |
| (50 mg/kg) | | | | |
| Methyltestosterone | 9.1 ± 0.4 | N.D. | N.D. | 0.60 ± 0.06 |
| (120 mg/kg) + | (n = 6) | | | (n = 6) |
| Folinic Acid | | | | |
| (50 mg/kg) | | | | |

N.D. = Not Determined

The results of these experiments indicate that rats administered DHEA or methyltestosterone daily for two weeks showed multi-organ depletion of adenosine. Depletion was dramatic in brain (60% depletion for DHEA, 34% for high dose methyltestosterone) and heart (37% depletion for DHEA, 22% depletion for high dose methyltestosterone). Co-administration of folinic acid completely abrogated steroid-mediated adenosine depletion. Folinic acid administered alone induce increase in adenosine levels for all organs studied.

### Example 3

### Preparation of the Experimental Model

Cell cultures, HT-29 SF cells, which represent a subline of HY-29 cells (ATCC, Rockville, Md.) and are adapted for growth in completely defined serum-free PC-1 medium (Ventrex, Portland, Me.), were obtained. Stock cultures were maintained in this medium at 37° (in a humidified atmosphere containing 5% CO₂). At confluence cultures were replated after dissociation using trypsin/EDTA (Gibco, Grand Island, N.Y.) and re-fed every 24 hours. Under these conditions, the doubling time for HT-29 SF cells during logarithmic growth was 24 hours.

### Example 4

### Flow Cytometry

Cells were plated at 10⁵/60-mm dish in duplicate. For analysis of cell cycle distribution, cultures were exposed to either 0, 25, 50, or 200 μM DHEA. For analysis of reversal of cell cycle effects of DHEA, cultures were exposed to either 0 or 25 μM DHEA, and the media were supplemented with MVA, CH, RN, MVA plus CH, or MVA plus CH plus RN or were not supplemented. Cultures were trypsinized following 0, 24, 48, or 74 hours and fixed and stained using a modification of a procedure of Bauer et al., Cancer Res., 46, 3173-3178 (1986). Briefly, cells were collected by centrifugation and resuspended in cold phosphate-buffered saline. Cells were fixed in 70% ethanol, washed, and resuspended in phosphate-buffered saline. One ml hypotonic stain solution [50 μg/ml propidium iodide (Sigma Chemical Co.), 20 μg/ml Rnase A (Boehringer Mannheim, Indianapolis, Ind.), 30 mg/ml polyethylene glycol, 0.1% Triton X-100 in 5 mM citrate buffer] was then added, and after 10 min at room temperature, 1 ml of isotonic stain solution [propidium iodide, polyethylene glycol, Triton X-100 in 0.4M NaCl] was added and the cells were analyzed using a flow cytometer, equipped with pulse width/pulse area doublet discrimination (Becton Dickinson Immunocytometry Systems, San Jose, Calif.) After calibration with fluorescent beads, a minimum of 2×10⁴ cells/sample were analyzed, data were displayed as total number of cells in each of 1024 channels of increasing fluorescence intensity, and the resulting histogram was analyzed using the Cellfit analysis program (Becton Dickinson).

### Example 5

### DHEA Effect on Cell Growth

Cells were plated 25,000 cells/30 mm dish in quadruplicate, and after 2 days received 0, 12.5, 25, 50, or 200 μM DHEA. Cell number was determined 0, 24, 48, and 72 hours later using a Coulter counter (model Z₁ Coulter Electronics, Inc. Hialeah, Fla.). DHEA (AKZO, Basel, Switzerland) was dissolved in dimethyl sulfoxide, filter sterilized, and stored at −20° C. until use.

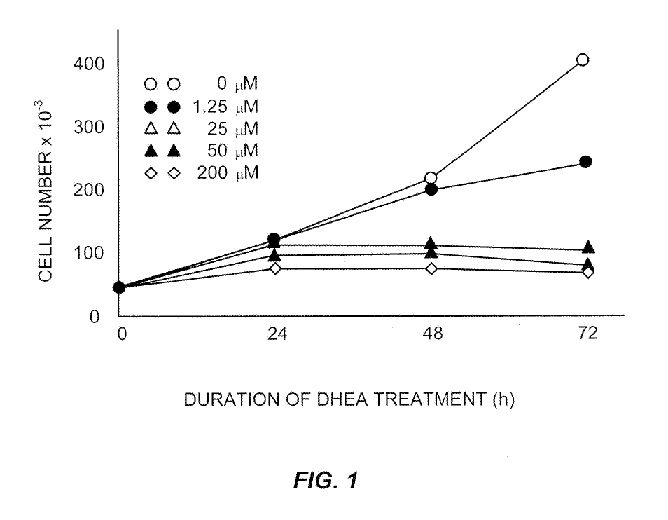

FIG. 1 illustrates the inhibition of growth for HT-29 cells by DHEA. Points refer to numbers of cells, and bars refer to SEM. Each data point was performed in quadruplicate, and the experiment was repeated three times. Where SEM bars are not apparent, SEM was smaller than symbol. Exposure to DHEA resulted in a reduced cell number compared to controls after 72 hours in 12.5 μM, 48 hours in 25 or 50 and 24 hours in 200 μM DHEA, indicating that DHEA produced a time- and dose-dependent inhibition of growth.

### Example 6

### DHEA Effect on Cell Cycle

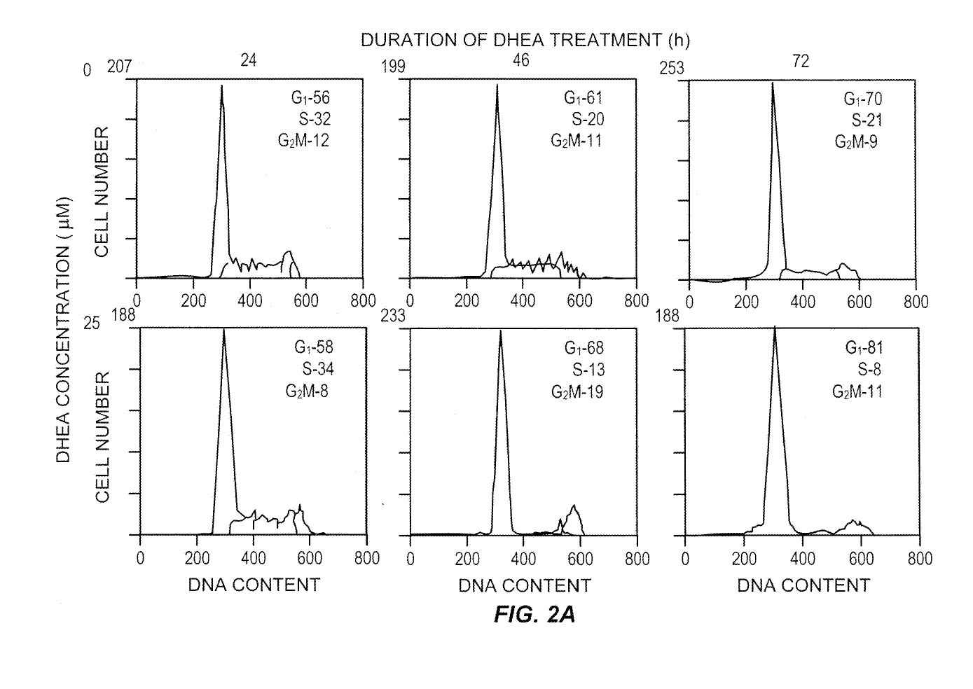

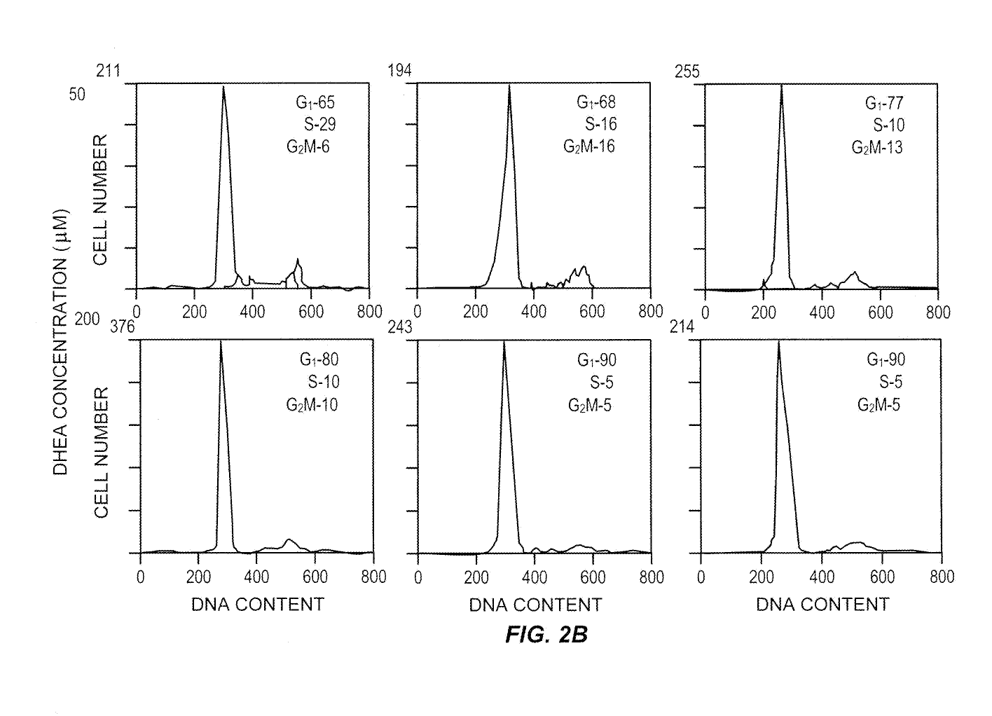

To examine the effects of DHEA on cell cycle distribution, HT-29 SF cells were plated (10⁵ cells/60 mm dish), and 48 hours later treated with 0, 25, 50, or 200 μM DHEA. FIG. 2 illustrates the effects of DHEA on cell cycle distribution in HT-29 SF cells. After 24, 48, and 72 hours, cells were harvested, fixed in ethanol, and stained with propidium iodide, and the DNA content/cell was determined by flow cytometric analysis. The percentage of cells in G₁, S, and G₂M phases was calculated using the Cellfit cell cycle analysis program. S phase is marked by a quadrangle for clarity. Representative histograms from duplicate determinations are shown. The experiment was repeated three times.

The cell cycle distribution in cultures treated with 25 or 50 μM DHEA was unchanged after the initial 24 hours. However, as the time of exposure to DHEA increased, the proportion of cells in S phase progressively decreased, and the percentage of cells in G₁, S and G₂M phases was calculated using the Cellfit cell cycle analysis program. S phase is marked by a quadrangle for clarity. Representative histograms from duplicate determinations are shown. The experiment was repeated three times. The cell cycle distribution in cultures treated with 25 or 50 μM DHEA was unchanged after the initial 24 hours. However, as the time of exposure to DHEA increased, the proportion of cells in S phase progressively decreased and the percentage of cells in G₁ phase was increased after 72 hours. A transient increase in G₂M phase cells was apparent after 48 hours. Exposure to 200 μM DHEA produced a similar but more rapid increase in the percentage of cells in G₁ and a decreased proportion of cells in S phase after 24 hours, which continued through the treatment. This indicates that DHEA produced a G₁ block in HT-29 SF cells in a time- and dose-dependent manner.

### Example 7

### Reversal of DHEA-Mediated Effect on Growth & Cell Cycle

Reversal of DHEA-mediated Growth Inhibition. Cells were plated as above, and after 2 days received either 0 or 25 μM DHEA-containing medium supplemented with mevalonic acid (“MVA”; mM) squalene (SQ; 80 μM), cholesterol (CH; 15 μg/ml), MVA plus CH, ribonucleosides (RN; uridine, cytidine, adenosine, and guanosine at final concentrations of 30 μM each), deoxyribonucleosides (DN; thymidine, deoxycytidine, deoxyadenosine and deoxyguanosine at final concentrations of 20 μM each). RN plus DN, or MVA plus CH plus RN, or medium that was not supplemented. All compounds were obtained from Sigma Chemical Co. (St. Louis, Mo.) Cholesterol was solubilized in ethanol immediately before use. RN and DN were used in maximal concentrations shown to have no effects on growth in the absence of DHEA.

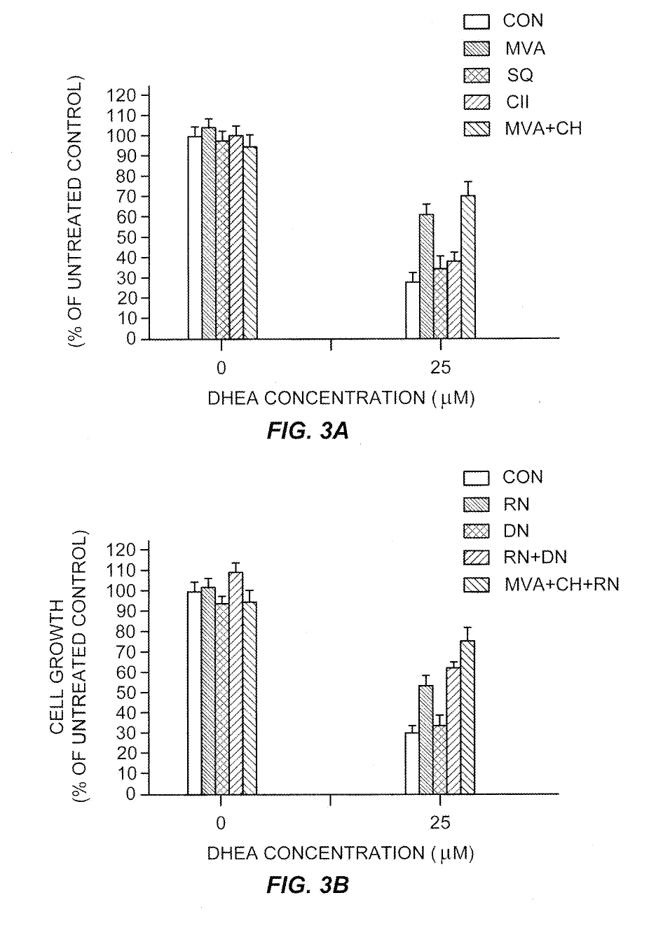

FIG. 3 illustrates the reversal of DHEA-induced growth inhibition in HT-29 SF cells. In A, the medium was supplemented with 2 μM MVA, 80 μM SQ, 15 μg/mt CH, or MVA plus CH (MVA+CH) or was not supplemented (CON). In B, the medium was supplemented with a mixture of RN containing uridine, cytidine, adenosine, and guanosine in final concentrations of 30 μM each; a mixture of DN containing thymidine, deoxycytidine, deoxyadenosine and deoxyguanosine in final concentrations of 20 μM each; RN plus DN (RN+DN); or MVA plus CH plus RN (MVA+CH+RN). Cell numbers were assessed before and after 48 hours of treatment, and culture growth was calculated as the increase in cell number during the 48 hour treatment period. Columns represent cell growth percentage of untreated controls; bars represent SEM. Increase in cell number in untreated controls was 173,370″6518. Each data point represents quadruplicate dishes from four independent experiments. Statistical analysis was performed using Student's t test χ p<0.01; ψp<0.001; compared to treated controls. Note that supplements had little effect on culture growth in absence of DHEA.

Under these conditions, the DHEA-induced growth inhibition was partially overcome by addition of MVA as well as by addition of MVA plus CH. Addition of SQ or CH alone had no such effect. This suggest that the cytostatic activity of DHEA was in part mediated by depletion of endogenous mevalonate and subsequent inhibition of the biosynthesis of an early intermediate in the cholesterol pathway that is essential for cell growth. Furthermore, partial reconstitution of growth was found after addition of RN as well as after addition of RN plus DN but not after addition of DN, indicating that depletion of both mevalonate and nucleotide pools is involved in the growth-inhibitory action of DHEA. However, none of the reconstitution conditions including the combined addition of MVA, CH, and RN completely overcame the inhibitory action of DHEA, suggesting either cytotoxic effects or possibly that additional biochemical pathways are involved.

### Example 8

### Reversal of DHEA Effect on Cell Cycle

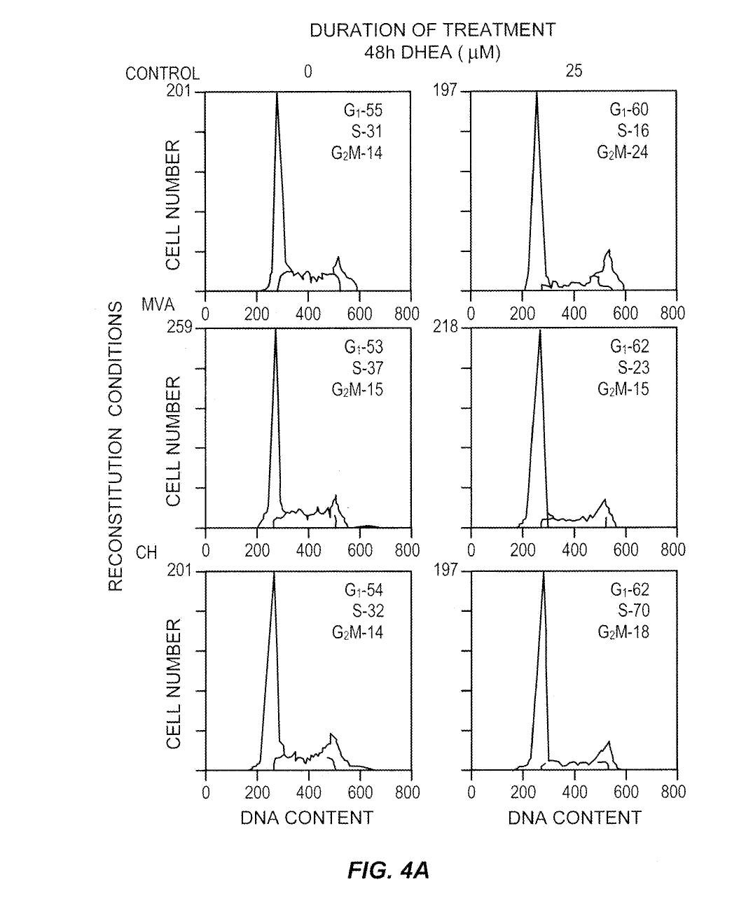

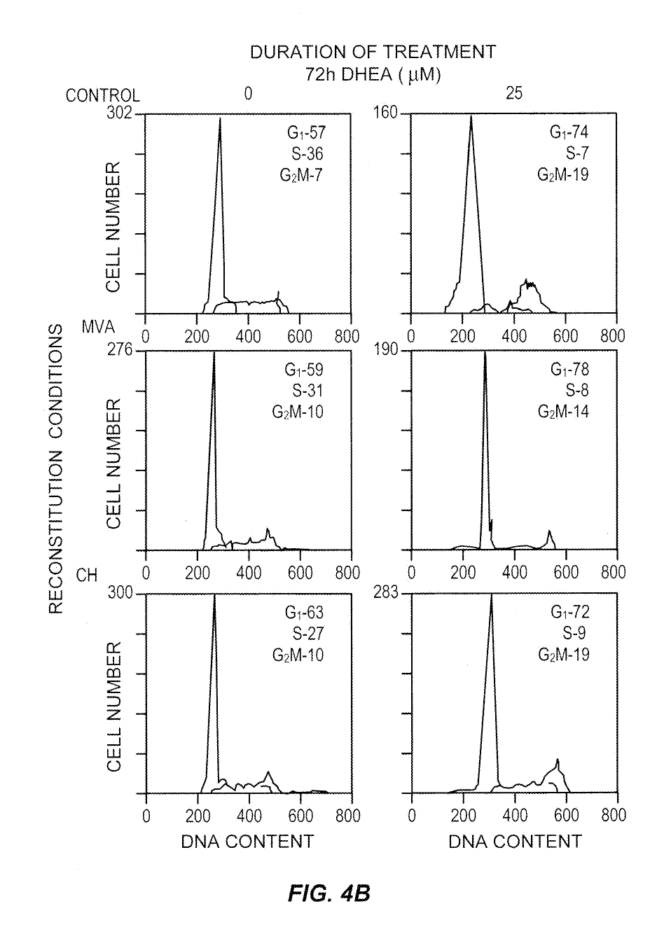

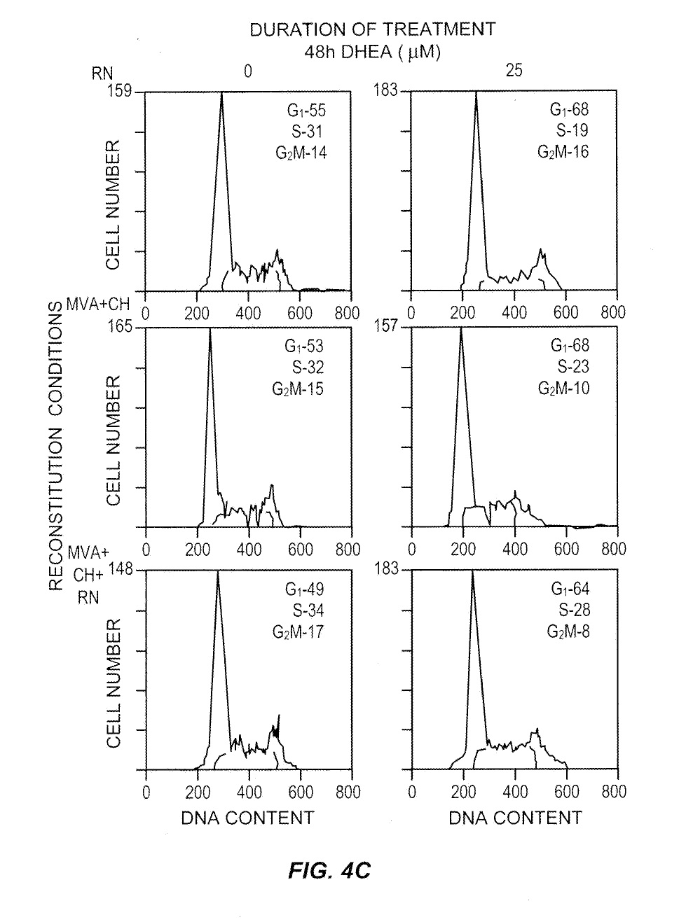

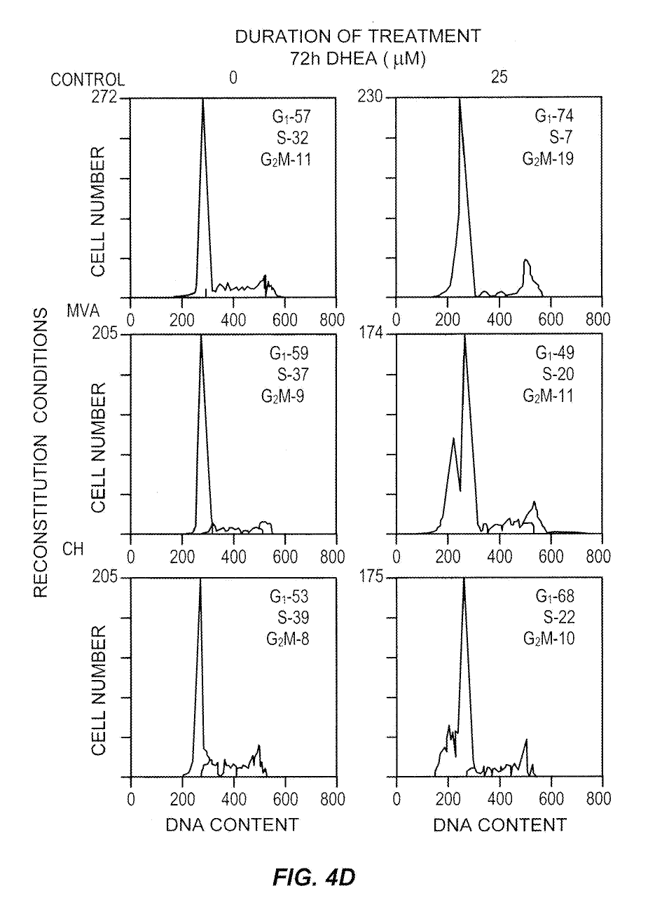

HT-29 SF cells were treated with 25 FM DHEA in combination with a number of compounds, including MVA, CH, or RN, to test their ability to prevent the cell cycle-specific effects of DHEA. Cell cycle distribution was determined after 48 and 72 hours using flow cytometry. FIG. 4 illustrates reversal of DHEA-induced arrest in HT-29 SF cells. Cells were plated (10⁵ cells/60 mm dish) and 48 hours later treated with either 0 or 25 FM DHEA. The medium was supplemented with 2 FM MVA; 15 Fg/ml CH; a mixture of RN containing uridine, cytidine, adenosine, and guanosine in final concentrations of 30 FM; MVA plus CH (MVA+CH); or MVA plus CH plus RN (MVA+CH+RN) or was not supplemented. Cells were harvested after 48 or 72 hours, fixed in ethanol, and stained with propidium iodine, and the DNA content per cell was determined by flow cytometric analysis. The percentage of cells in G₁, S, and G₂M phases were calculated using the Cellfit cell cycle profile analysis program. S phase is marked by a quadrangle for clarity. Representative histograms from duplicative determinations are shown. The experiment was repeated two times. Note that supplements had little effect on cell cycle progression in the absence of DHEA.

With increasing exposure time, DHEA progressively reduced the proportion of cells in S phase. While inclusion of MVA partially prevented this effect in the initial 48 hours but not after 72 hours, the addition of MVA plus CH was also able to partially prevent S phase depletion at 72 hours, suggesting a requirement of both MVA and CH for cell progression during prolonged exposure. The addition of MVA, CH, and RN was apparently most effective at reconstitution but still did not restore the percentage of S phase cells to the value seen in untreated control cultures. CH or RN alone had very little effect at 48 hours and no effect at 72 hours. Morphologically, cells responded to DHEA by acquiring a rounded shape, which was prevented only by the addition of MVA to the culture medium (data not shown). Some of the DNA histograms after 72 hours DHEA exposure in FIG. 4 also show the presence of a subpopulation of cells possessing apparently reduced DNA content. Since the HT-29 cell line is known to carry populations of cells containing varying numbers of chromosomes (68-72; ATCC), this may represent a subset of cells that have segregated carrying fewer chromosomes.

### Example 9

### Conclusions

The examples above provide evidence that in vitro exposure of HT-29 SF human colonic adenocarcinoma cells to concentrations of DHEA known to deplete endogenous mevalonate results in growth inhibition and G, arrest and that addition of MVA to the culture medium in part prevents these effects. DHEA produced effects upon protein isoprenylation which were in many respects similar to those observed for specific 3-hydroxy-3-methyl-glutaryl-CoA reductase inhibitors such as lovastatin and compactin. Unlike direct inhibitors of mevalonate biosynthesis, however, DHEA mediates its effects upon cell cycle progression and cell growth in a pleiotropic manner involving ribo- and deoxyribonucleotide biosynthesis and possibly other factors as well.

### Example 10

### Ipratropium Bromide 0.03% (IB) & Beclomethasone Dipropionate Comparison

Thirty-three children with non-allergic perennial rhinitis (NAPR) and 113 with allergic perennial rhinitis (APR) were randomly assigned to either IB or BDP for 6 months in a single-blind, multicenter protocol in which the physicians were blinded to treatment. At each visit, patients and physicians rated symptom control of rhinorrhea, nasal congestion, and sneezing. Patients also completed quality of life questionnaires at baseline and after 6 months of therapy. Both treatments showed a significant improvement in control of rhinorrhea, congestion, and sneezing compared with baseline over the 6 months of treatment (P<0.05). Only for the control of sneezing was BDP consistently better than IB (P<0.05). Among the patients given IB, 61% to 73% assessed the control of rhinorrhea as good or excellent on different study visit days, 43% to 60% similarly rated the control of nasal congestion, and 39% to 43% the control of sneezing. The results for BDP were 68% to 78% for the control of rhinorrhea, 55% to 72% for the control of nasal congestion, and 54% to 68% for the control of sneezing. Quality of life assessment documented that both drugs significantly reduced interference with daily activities and disturbance of mood due to rhinorrhea, compared with baseline (P<0.05). Both treatments were well tolerated with IB causing less nasal bleeding and irritation than BOP. Ipratropium bromide was safe and effective in controlling rhinorrhea and diminishing the interference by rhinorrhea in school attendance, concentration on school work, and sleep. Ipratroprium bromide was as effective as BDP in the control of rhinorrhea and showed a relatively good effect on congestion. Patient and physician assessment favored BDP in the control of sneezing. Milgrom et al. Ann. Allergy Asthma Immunol. 83(2): 105-11 (1999). In Examples 11 to 16, micronized DHEA and micronized anti-muscarinic agent (as the hydroxynaphthoate) are added in the proportions given below either dry or after predispersal in a small quantity of stabilizer, disodium dioctylsulphosuccinate, lecithin, oleic acid or sorbitan trioleate/trichloro-fluoromethane solution to a suspension vessel containing the main bulk of the trichlorofluoromethane solution. The resulting suspension is further dispersed by an appropriate mixing system using, for example, a high shear blender, ultrasonics or a microfluidizer until an ultrafine dispersion is created. The suspension is then continuously recirculated to suitable filling equipment designed for cold fill or pressure filling of dichlorodifluoromethane. The suspension may be also prepared in a suitable chilled solution of stabilizer, in trichlorofluoromethane/difluoromethane.

### Example 11

### Use of Ipratropium Bromide in Acute Asthma Exacerbation in Adults and Children

Following is a summary of the physiological actions of ipratropium and its use as an anti-cholinergic bronchodilator. Evidence available from randomized trials and from two meta-analyses is summarized to determine whether the addition of inhaled ipratropium to inhaled beta 2-agonist therapy is effective in the treatment of acute asthma exacerbation in small children and adults. There were published reports of randomized, controlled trials assessing the use of ipratropium and concurrent beta 2-agonists in adult acute asthma exacerbation. Data from 10 studies of adult asthmatics, reporting on a total of 1,377 patients, were pooled in a meta-analysis using a weighted-average method. The use of nebulized ipratropium/beta2-agonist combination therapy was associated with a pooled 7.3% improvement in forced expiratory volume in 1 sec (95% confidence interval (CI), 3.8-10.9%), and a 22.1% improvement in peak expiratory flow (95% CI, 11.0-33.2%), when compared with patients who received only beta 2-agonist without ipratropium. For the three trials in adults reporting hospital admission data (n=1064), adult patients receiving ipratropium had a relative risk of hospitalization of 0.80 (95% CI, 0.61-1.06). Similarly, randomized controlled studies of pediatric asthma exacerbation and a meta-analysis of pediatric asthma patients suggest that ipratropium added to beta 2-agonists improved lung function and also decreased hospitalization rates, especially among children with severe exacerbations of asthma.

Neither the adult nor the pediatric studies reported severe adverse effects attributable to ipratropium when it was used in conjunction with beta 2-agonists.

In conclusion, there is a modest statistical improvement in airflow obstruction when ipratropium is used as an adjunctive to beta 2-agonists for the treatment of acute asthma exacerbation. In pediatric asthma exacerbation, the use of ipratropium also appears to improve clinical outcomes. This, however, has not been definitively established in adults. The use of ipratropium/beta 2-agonist combination therapy in acute asthmatic exacerbation has been recommended because the addition of ipratropium provides physiological evidence of benefit without risk of adverse effects. Aaron S. D., J. Asthma 38(7):521-30 (2001).

### Example 12

### Effect of Tiotropium Bromide on COPD Patients*

Tiotropium bromide is a new long-lasting anticholinergic drug which, like ipratropium bromide, is a quaternary ammonium derivative. It binds with high affinity to muscarinic receptors but dissociates very slowly from M(1)- and M(3)-muscarinic receptors. Pharmacology studies have demonstrated a prolonged protective effect against cholinergic agonists and cholinergic nerve stimulation in animal and human airways. In Phase II studies single inhaled doses of tiotropium bromide have a bronchodilator and bronchoprotective effect in asthmatic and chronic obstructive pulmonary disease (COPD) patients of over 24 h. In Phase III studies, once daily inhaled tiotropium is an effective bronchodilator in COPD patients, giving great improvement in lung function and reduction in symptoms than ipratropium bromide given four times daily. The drug is well-tolerated and the only side effect of note is dryness of the mouth which occurs in approximately 10% of patients. Since, anticholinergics are the bronchodilators of choice in COPD it is likely that tiotropium bromide will become the most widely used bronchodilator for COPD patients in the future. Barnes, P. J., Expert Opin. Investing. Drugs 10(4): 733 (2001).

### Example 13

### Ipratropium & Albuterol Effect on COPD Patients

To determine whether the combination of ipratropium bromide and albuterol results in greater and more consistent pulmonary function test (PFT) response rates than ipratropium bromide or albuterol alone in patients with COPD, DESIGN: Retrospective review of two recently completed 3-month, randomized, double-blind, parallel, multicenter, phase III trials. SETTING: Outpatient. PATIENTS: A total of 1,067 stable patients with COPD. INTERVENTIONS: Ipratropium bromide (36 microg qid), albuterol base (180 microg qid), or an equivalent combination of ipratropium bromide and albuterol sulfate (42 microg and 240 microg qid, respectively). MEASUREMENTS AND RESULTS: PFT response rates were analyzed using 12% and 15% increases in FEV1 compared with baseline values and were measured in the various treatment groups on days 1, 29, 57, and 85 in these trials. Regardless of whether a 12% or a 15% increase in FEV1 was used to define a positive response, an equivalent combination of ipratropium bromide and albuterol sulfate was superior to the individual agents (p<0.05; all comparisons within 30 min). In addition, a 15% or more increase in FEV1 was seen in >80% of patients who received the combination of ipratropium and albuterol sulfate during the initial PFT and continued to be observed 3 months after initial testing. CONCLUSIONS: Use of a combination of ipratropium bromide and albuterol sulfate is superior to the individual agents in identifying PFT reversibility in patients with COPD.

Dorinsky et al., Chest 115(4): 966-71 (1999).

### Example 14

### Anti-Muscarinic Agent

### Effect on COPD & Allergic Rhinitis

Antimuscarinic treatment of airway disease is used as an effective bronchodilator in chronic obstructed pulmonary disease (COPD) as well as an antisecretory drug for watery rhinorrhea (Allergic Rhinitis). Present formulations are limited to ipratropium bromide, a safe and effective respiratory therapeutic. Ipratropium has been documented by spirometry, as an effective bronchodilator both alone and in combination with albuterol. The evidence suggests that anticholinergics can affect other important aspects of COPD, such as dynamic hyperinflation.

Witek, T J. Jr., Respir. Case Clin. N. Am 5 (4): 521-36 (1999).

### Example 16

### Metered Dose Inhaler

| Active Ingredient | Target per Actuation | |
|---|---|---|
| Ipratropium Bromide | 25.0 | μg |
| DHEA | 400 | mg |
| Stabilizer | 5.0 | μg |
| Solvent 1 | 23.70 | mg |
| Solvent 2 | 61.25 | mg |

### Example 17

### Metered Dose Inhaler

| Active Ingredient | Target per Actuation | |
|---|---|---|
| Ipratropium Bromide | 25.0 | μg |
| DHEA-S | 400 | mg |
| Stabilizer | 7.5 | μg |
| Solvent 1 | 23.67 | mg |
| Solvent 2 | 61.25 | mg |

### Example 18

### Metered Dose Inhaler

| Active Ingredient | Target per Actuation | |
|---|---|---|
| Tiotropium Bromide | 25.0 | μg |
| DHEA | 400.0 | mg |
| Stabilizer | 15.0 | μg |
| Solvent 1 | 23.56 | mg |
| Solvent 2 | 61.25 | mg |

### Example 19

### Metered Dose Inhaler

| Active Ingredient | Target per Actuation | |
|---|---|---|
| Tiotropium Bromide | 25.0 | μg |
| DHEA-S | 400.0 | mg |
| Stabilizer | 15.0 | μg |
| Solvent 1 | 23.56 | mg |
| Solvent 2 | 61.25 | mg |

In the following Examples 20 to 23, the active ingredients are micronized and bulk blended with lactose in the proportions given above. The blend is filled into hard gelatin capsules or cartridges or into specifically constructed double foil blister packs (Rotadisks blister packs, Glaxo® to be administered by an inhaler such as the Rotahaler inhaler (Glaxo®) or in the case of the blister packs with the Diskhaler inhaler (Glaxo®).

### Example 20

### Metered Dose Dry Powder Formulation

| Active Ingredient | Per Cartridge or Blister | |
|---|---|---|
| Ipratropium Bromide | 72.5 | μg |
| DHEA | 1.00 | mg |
| Lactose Ph. Eur. | to 12.5 or 25.0 | mg |

### Example 21

### Metered Dose Dry Powder Formulation

| Active Ingredient | Per Cartridge or Blister | |
|---|---|---|
| Ipratropium Bromide | 72.5 | μg |
| DHEA-S | 1. | mg |
| Lactose Ph. Eur. | to 12.5 or 25.0 | mg |

### Example 22

### Metered Dose Dry Powder Formulation

| Active Ingredient | Per Cartridge or Blister | |
|---|---|---|
| Tiotropium Bromide | 72.5 | μg |
| DHEA | 1 | mg |
| Lactose Ph. Eur. | to 12.5 or 25.0 | mg |

### Example 23

### Metered Dose Dry Powder Formulation

| Active Ingredient | Per Cartridge or Blister | |
|---|---|---|
| Tiotropium Bromide | 72.5 | μg |
| DHEA-S | 1 | mg |
| Lactose Ph. Eur. | to 12.5 or 25.0 | mg |

### Example 24

### Effect of DHEA-S on Pulmonary Function & Inflammation

This study defines the effect of 5 days of treatment with DHEA-S on allergen-induced early phase response, and pulmonary inflammation in dust mite-sensitized cynomolgus monkeys. Baseline parameters were measured the week prior to treatment. DHEA-S (5 mg/ml, 2 ml nebulized) or an equivalent volume of saline was delivered once per day via nebulization (Pari LC+) through a canine facemask covering the nose and mouth on days 1 through 5. Allergen challenge and pulmonary function testing were performed on day 3 several hours after drug treatment, and broncho-alveolar lavage fluid BALF) was collected 48 hours post-challenge. The study includes 6 animals studied under a crossover design with a minimum of 4 weeks rest between study arms. The treatment with DHEA-S led to the following changes in the disease status of the animals:

Lung compliance improved during the early phase response. See, FIG. 4.3.1.1. The treatment with DHEA-S attenuated the drop in airway compliance following allergen challenge by 87% as compared to saline treatment. The total area under the curve for the change in compliance following allergen challenge was −49±116 in the DHEA-S treated group vs. −394±127 in the saline control group, p=0.035. Lung resistance improved during the early phase response. See, FIG. 4.3.1.2. Treatment with DHEA-S attenuated the increase in airway resistance following allergen challenge by 90% as compared to saline treatment. The total area under the curve for the change in resistance following allergen challenge was 54±90 in the DHEA-S treated group vs. 564±266 in the saline control group, p=0.045. The treatment with DHEA-S decreased % eosinophils in BALF 48 hours after allergen challenge by approximately 57%, p=0.012, as compared to saline. See, FIG. 4.3.1.3. Moreover, treatment with DHEA-S decreased the percentage of neutrophils in BALF 48 hours after allergen challenge by 42%, p=0.008, as compared to saline. See, FIG. 4.3.1.3.

These results show that nebulized DHEA-S attenuates inflammation and counters the decline in pulmonary function normally induced by aeroallergen challenge.

### (A) Basal Allergic Responsiveness

### Allergen-Induction of Inflammation

Dust mite challenge induced an increase in the percentage of eosinophils in the BAL fluid 48 hours after aerosol administration of allergen. Eosinophils increased from 0.25%±0.17% to 4.8%±1.4% (P-0.018) in the saline control group confirming that the allergen induced migration of eosinophils into the airways.

### Pulmonary Function Response to Allergen

Allergen challenge following saline treatment induced a significant early phase response as indicated by an average decrease in compliance of 30% and increase in resistance of 50% induced by dust mite at 1:100 dilution. These data confirm that the animals in the study responded to the relevant antigen with airway inflammation and altered pulmonary function.

### (B) Effects of PHBA-S on Pulmonary Function

### Early Phase Responses to Dint Mite

Effects on airway obstruction were measured by changes in dynamic compliance in response to allergen challenge. Allergic Cynomolgus monkeys were monitored for 15 minutes following allergen challenge and the values plotted against time. Changes in dynamic compliance were determined by the % change in compliance as compared to the animal's initial saline value recorded prior to challenge. Changes in response to allergen and/or treatment were calculated using the area under the curve (AUC) where an AUC unit is defined as % change×minutes. Treatment with DHEA-S attenuated the drop in airway compliance following allergen challenge by 87% as compared to saline treatment. With saline treatment, the total AUC was −394±127 units compared to −49±116 units for animals treated with DHEA-S. See, FIGS. 4.3.1.1; 4.3.1.2. This difference in the total AUC for vehicle and DHEA-S treated animals was determined to be significant using Student's t-test for paired observations, p=0.035. Likewise, treatment with DHEA-S attenuated the increase in airway resistance following allergen challenge by 90% as compared to saline treatment. The AUC. for resistance was significantly greater with saline treatment than DHEA-S treatment (564±266 vs. 54±90 units) (P=0.045).

### (C) Effects of DHEA-S on Inflammation

### BALF Cell Count

Treatment with DHEA-S tended to decrease the total number of leukocytes migrating into the BALF 48 hours after allergen challenge. See, FIG. 4.3.1.3. Leukocytes rose from 31.4×10⁴ to 54.9×10⁴ cell/ml with saline treatment versus a rise from 34.5×10⁴ to 39.4×10⁴ cell/ml in the DHEA-S treatment group. Whereas the percentage of eosinophils in the saline treated group increased after allergen exposure, the percentage of eosinophils in the DHEA-S treated animals was not significantly different before and after allergen challenge. See, FIG. 4.3.1.3. Eosinophil numbers (cells/ml) in the BALP 48 hours after allergen challenge were approximately 64% less following treatment with DHEA-S (1.6±0.8×10⁴ cells/ml) compared to treatment with saline (4.3±1.7×10⁴ cells/ml), but the differences did not reach significance. Following allergen exposure, the percentage of neutrophils in the BALF was significantly elevated in the saline group, p-0.037. DHEA-S treatment attenuated the neutrophil influx to a level that was not significantly higher than pre-challenge levels. See, FIG. 4.3.1.3. The absolute number of neutrophils (cells/ml) in the saline group increased to 22.0±30.3×10⁴ cells/ml while they raised to only 9.7±2.2×10⁴ cells/ml with DHEA-S treatment.

### Conclusions

The results of this study provide evidence that DHEA-S attenuates the inflammatory effects of inhaled allergen in allergic cynomolgus monkeys. An inhibition of eosinophilic inflammation as well as neutrophil influx, is indicated. An inhibition of the early phase response to allergen challenge is also demonstrated. These observations are consistent with data derived from the allergic rabbit model.

### Example 25

### DHEA-S Effect on Allergen-Induced Airway Obstruction & Pulmonary Inflammation

This study defines the effect of 7 days of treatment with DHEA-S on allergen-induced bronchial hyperresponsiveness (BHR), early and late phase responses, and pulmonary inflammation in dust mite-sensitized rabbits. Baseline BHR and inflammation were first determined. The following week, allergen challenge, pulmonary function testing and BALF were performed on the untreated rabbits. Histamine sensitivity was determined 24 hours post allergen challenge. Rabbits were rested for 3 weeks then baseline values were re-established. The following week, DHEA-S (5 mg/day) was delivered once a day for 7 days via an intratracheal powder injector through the endrotracheal tube of the anesthetized rabbit. On day 8, 24 hrs after the last treatment, allergen challenge, pulmonary function testing and BALF were performed as previously. On day 9, 24 hrs after allergen challenge, a histamine challenge was performed. The drug was not micronized prior to use thus only a portion of the administered date was expected to reach the deep lung. The study includes 4 animals studied under a crossover design. The treatment with DHEA-S led to changes in the disease status of the animals that are described in the following paragraph.

Lung compliance improved during the early and late phase responses. See FIG. 4.3.2.1. Treatment with EP 1-12312 attenuated the drop in airway compliance following allergen challenge by 86% as compared to allergen alone. The total area under the curve for the change in compliance following allergen challenge was −20±23 in the DHEA-S treated group vs. −146±29 in the control group, p=0.036. Lung resistance improved during the early and late phase responses. See, FIG. 4.3.2.1. The treatment with EPI-12312 attenuated the increase in airway resistance following allergen challenge by 54% as compared to allergen alone. The total area under the curve for the change in resistance following allergen challenge was 495±341 in the DHEA-S treated group vs. 1069±243 in the control group though the difference did not reach significance because of the large variation in a small number of animals. Moreover, there was a moderate modification associated with DHEA-S of cell infiltrate into the BAL fluid after challenge with dust mite. Finally, a slight mean inhibition of eosinophils was observed at 24 hours, and a prominent inhibition of neutrophils was observed at 15 minutes (10.2 vs. 1.3×10⁴ cells/ml) and 6 hours (43.8 vs. 32.2×10⁴ cells/ml) after challenge. In addition, there was a downward shift in the 24-hour histamine responsiveness following allergen challenge in the DHEA-S treatment group.

These results indicate that DHEA-S, delivered as a powder, attenuated inflammation and the decline in pulmonary function normally induced by aeroallergen challenge.

### (A) Basal Allergic Responsiveness

### Allergen-Induction of Inflammation

Dust mite challenge induced a significant increase in the number of eosinophils, neutrophils, and macrophages in the BAL fluid 6 hours after aerosol administration of allergen. The percentage of eosinophils also increased from less than 0.2% to 6% six hours after dust mite challenge in the vehicle control animals confirming that the allergen induced migration of eosinophils into the airways.

### Pulmonary Function

PC₄₀ for adenosine in the 4 animals ranged from 0.55 to 4.56 mg/ml with mean of 2.5 mg/ml. In the vehicle control group, dust mite exposure decreased peak compliance recorded during the early phase by 45% relative to saline, and 28% in the late phase compared to saline, respectively. Dust mite exposure increased maximum airway resistance by 234% and 290% during the early and late phase responses, respectively. These data confirm that the animals in the study were sensitive to aerosolized adenosine and responded to the relevant antigen with airway inflammation and altered pulmonary function.

### (B) Effects OP DHEA-S on Pulmonary Function

### Effect of Dust Mite on Compliance

Effects on airway obstruction were measured by changes in dynamic compliance and total lung resistance in response to allergen challenge. Rabbits were monitored for 6 hours following allergen challenge and the values plotted against time. Changes in dynamic compliance were determined by the % change in compliance as compared to the animal's initial saline value recorded prior to challenge. Changes in response to allergen and/or treatment were calculated using the area under the curve (AUC) where an AUC unit is defined as % change×hour. Treatment with DHEA-S attenuated the drop in airway compliance following allergen challenge by 86% as compared to allergen alone. Following allergen alone the total AUC was −146 units compared to −20 units for animals treated with DHEA-S. This difference in the total AUC for vehicle and DHEA-S treated animals was determined to be significant using Student's t-test for paired observations, p=0.036. Similarly, treatment with DHEA-S attenuated the increase in airway resistance following allergen challenge by 54% as compared to allergen alone. The difference in the AUC during the early phase response following allergen alone and DHEA-S treatment was significant, at p=0.023, using a paired t test. Variability during the late phase kept differences in AUC between the vehicle and drug treatment groups from being significant when analyzed using a paired t-test.

### Effect of Dust Mite on Resistance and Histamine Responsiveness

Administration of DHEA-S reduced the airway resistance changes in the 6 hours after aerosol dust mite challenge by over 200%. The total AUC for the control and treatment groups was 1069 vs. 495. However, due to variability within the small number of animals statistical significance was not reached. Treatment with DHEA-S shifted the hyperresponsiveness response to histamine. The histamine response in the saline treated group was greater after dust mite and more variable than in the DHEA-S treatment group.

### (C) DHEA-S Effects on Inflammation

### BALF Cell Counts

Inflammatory cell migration info the BAL fluid was reduced by DHEA-S treatment. Animals treated with DHEA-S exhibited lower numbers of eosinophils (2.0 vs. 3.2×10⁴ cells/ml) and macrophages (17.8 vs. 24.1×10⁴ cells/ml) in BALF 24 hours after allergen challenge compared to the control group. There was a reduction in neutrophils (1.3 vs. 10.2×10⁴ cells/ml) and macrophages (19.4 vs. 29.6×10⁴ cells/ml) at 15 minutes after dust mite challenge. This reduction persisted through the 6 hours time point but was less pronounced. It should be noted that this 24 hour post-dust mite time point was 48 hours after the last treatment with DHEA-S.

### Conclusions

Although there was a marked inhibition of both early and late phase responses of between 50 and 93%, the statistical differences were only significant for the early phase. There was an obvious improvement in both compliance and resistance following administration of DHEA-S prior to the dust mite challenge, and a slight modification of histamine responsiveness. There was a trend of reduction in inflammatory cells following dust mite challenge with reductions in eosinophils, neutrophils and macrophages. These results are likely to be improved using micronized formulations to ensure optimized delivery to the deep lung.

### Example 26

### DHEA-S Effect on Allergen-Induced Airway Obstruction & Pulmonary Inflammation

This study defines the effect of 7 days of treatment with DHEA-S on allergen-induced bronchial hyperresponsiveness (BHR), early and late phase responses, and pulmonary inflammation in dust mite-sensitized rabbits. Baseline BHR and inflammation were first determined. DHEA-S (5 mg/ml, 2 ml nebulized) or an equivalent volume of vehicle was delivered once per day via nebulization through a pediatric facemask covering the nose and mouth on days 1 through 7. On day 8, 24 hours after the last treatment, allergen challenge, pulmonary function testing and BALF were performed. On day 9, 24 hours after allergen challenge a histamine challenge was performed and the lungs removed for histology. The study includes 5 animals studied under a crossover design. There was a 3 week resting period between study arms and lungs were taken during the second treatment arm only. The treatment with DHEA-S led to the changes in the disease status of the animals described in the following paragraph.

Lung compliance improved during the early and late phase responses. See, FIG. 4.3.3.1. Treatment with DHEA-S attenuated the drop in airway compliance following allergen challenge by 62% as compared to vehicle treatment. The total area under the curve for the change in compliance following allergen challenge was +38±27 in the DHEA-S treated group vs. −99±11 in the control group, P-0.02. DHEA-S also decreased the number of inflammatory cell migration into the BAL fluid by 68% (FIG. 4.3.3.2). The animals treated with DHEA-S exhibited lower numbers of eosinophils (52%), neutrophils (69%), and macrophages (68%) in BALF 6 hours after allergen-challenge compared to the vehicle control group, p<0.01. Inflammation of the airway wall decreased. See, FIGS. 4.3.3.3 through 4.3.3.6 attached to this patent. Histological evaluation by a veterinary pathologist blinded to the group designations revealed that DHEA-S treatment reduced inflammatory cell cuffing of the vessels and bronchi, bronchus-associated lymphoid tissue hyperplasia, and epithelial sloughing.

These results indicate that nebulized DHEA-S attenuated inflammation and the decline in pulmonary function normally induced by aeroallergen challenge.

### (A) Basal Allergic Responsiveness

### Allergen-Induced Inflammation

Dust mite challenge induced a significant increase in the number of eosinophils, neutrophils, and macrophages in the BAL fluid six hours after aerosol administration of allergen. The percentage of eosinophils also increased from less than 0.2% to 8% six hours after dust mite challenge in the vehicle control animals confirming that the allergen induced migration of eosinophils into the airways.

### Pulmonary Function

PC₄₀ for adenosine in the 5 animals ranged from 2.0 to 8.4 mg/ml with mean of 4.5 mg/ml. In the vehicle control group, dust mite exposure decreased peak compliance recorded during the early phase by 32% relative to saline, and 21% in the late phase compared to saline, respectively. Dust mite exposure increased maximum airway resistance by 64% and 82% during the early and late phase responses, respectively. These data confirm that the animals in the study were sensitive to aerosolized adenosine and responded to the relevant antigen with airway inflammation and altered pulmonary function.

### (B) Effects of DHEA-S on Pulmonary Function

### Dust Mite Challenge Effect Upon Compliance

Effects on airway obstruction were measured by changes in dynamic compliance and total lung resistance in response to allergen challenge. Rabbits were monitored for 6 hours following allergen challenge and the values plotted against time. Changes in dynamic compliance were determined by the % change in compliance as compared to the animal's initial saline value recorded prior to challenge. Changes in response to allergen and/or treatment were calculated using the AUC where an AUC unit is defined as % change×hour. Treatment with DHEA-S attenuated the drop in airway compliance following allergen challenge by 62% as compared to vehicle treatment. Following allergen alone the total AUC was −99 units compared to +38 units for animals treated with DHEA-S. This difference in the total AUC for vehicle and DHEA-S treated animals was determined to be significant using Student's t-test for paired observations, at p=0.022. Similarly, the difference in the AUC during the late phase response following vehicle and DHEA-S treatment was significant, at p=0.012, using a paired t test. Variability during the early phase kept differences in AUC between the vehicle and drug treatment groups from being significant when analyzed using a paired t-test. However, a trend analysis incorporating a polynomial model for analysis of repeated measures indicated that the shapes of the compliance curves during the early phase response were different for the vehicle control and drug treatment groups, at p=0.04.

### Resistance Response to Dust Mite & Histamine Responsiveness

Administration of DHEA-S had no significant effect on airway resistance changes in the 6 hours after aerosol dust mite challenge. The total AUC for the control and treatment groups was 315 vs. 377. DHEA-S had little effect upon resistance in this study. This may be due to particular aspects of the allergic rabbit model, which was designed to study compliance, or to the crossover design, it is worth pointing out that currently available steroids in use for asthma or COPD administered under similar circumstances would not be expected to correct allergen-induced increases in resistance. Likewise, treatment with DHEA-S did not improve hyperresponsiveness to histamine. The PC₅₀ for control and DHEA-S treated animals was 0.84 vs. 0.86 mg/ml. It should be noted that the histamine response was measured 48 hours after the last administration of DHEA-S.

### (C) DHEA-S Effect on Inflammation

### BALF Cell Count

Inflammatory cell migration into the BAL fluid was reduced by DHEA-S treatment. Animals treated with DHEA-S exhibited lower numbers of eosinophils, neutrophils, and macrophages in BALF 6 hours after allergen challenge compared to the vehicle control group, at p<0.01, when analyzed by ANOVA and Fisher's lest for LSD. Six hours after dust mite challenge, the BAL fluid from vehicle- and drug-treated animals contained 8×10⁴ vs. 4×10⁴ eosinophils/ml, 94×10⁴ vs. 29×10⁴ neutrophils/ml, 37×10⁴ vs. 12×10⁴ respectively. The effect was not significant 24 hours after dust mite challenge. It should be noted that this 24 hours post-dust mite time point was 48 hours after the last treatment with DHEA-S.

### Histology

Effects on interstitial inflammation were evaluated by histology. A veterinary pathologist evaluated hematoxylin and eosin stained sections in a blinded manner. Sections were scored for inflammatory parameters using a scale of 0 to 4 with 0 representing no finding and 4 representing severe. Lung sections from animals treated with DHEA-S exhibited less inflammation associated with the airways. All of the following parameters were scored lower in the DHEA-S treated lungs compared to the vehicle control group: cuffing of the airways and blood vessels by inflammatory cells, bronchial-associated lymphoid tissue hyperplasia, and bronchitis/bronchiolitis. Bronchitis/bronchiolitis encompassed denudation of the airway and intraepithelial inflammatory cells. Parameters associated with alveolar complications showed no consistent pattern.

### Effects of DHEA-S on Serum and BAL DHEA and DHEA-S Levels

No significant change in DHEA and DHEA-S concentrations in serum were observed with DHEA-S treatment. Prior to DHEA-S treatment serum samples exhibited DHEA and DHEA-S concentrations of 0.9 μg/ml and 4.7 μ.g/dl, respectively. Twenty-four hours after the last of seven daily treatments, DHEA and DHEA-S concentrations were 1.1 ng/ml and 4.9 μg/dl, respectively. DHEA-S was not detectable in BAL fluid. DHA concentrations could be measured, but no correlation with treatment was apparent.

### Observations Off DHEA-S Effects

Animals receiving 7 days of treatment with DHEA-S via the facemask tolerated the pulmonary function testing and histamine challenges better than vehicle-treated animals. The qualitative evidence supporting this statement is that animals recovered normal function more quickly following the procedures. Furthermore, there was no evidence of nasal congestion following histamine and dust mite challenges in the DHEA-S treated animals.

### Conclusion

Resistance and compliance are indicators of proximal airway and distal lung function, respectively. However, the two parameters are not completely independent and their measurement can be affected by respiratory rate and tidal volume. With that in mind, the finding that DHEA-S improves compliance during the early and late phase indicates that its main effect is to reduce the work of breathing during an asthmatic attack by increasing the ease with which the distal lung inflates.

### Example 27

### Effect of Inhaled DHEA-S, Pulmicort® & Saline on Allergen Induced Airway Obstruction & Inflammation

This follow-up study compared the effects of DHEA-S and Puimicort® (budesonide inhalation suspension) on allergen-induced bronchial hyperresponsiveness (BHR) to histamine and pulmonary inflammation in dust mite-sensitized rabbits. The study included 18 animals divided into 3 treatment groups: saline, DHEA-S, and Puimicort. The animals were treated for once per day for 5 consecutive days via nebulization through a pediatric facemask covering the nose and mouth. Treatments were DHEA-S, 10 mg/day (5 mg/ml), Pulmicort Respules, 0.5 mg/day (0.5 mg/2 ml, note: this is the standard human dose) or saline (2 ml). A dust mite challenge was performed on day 4, and development of airway inflammation was assessed 6 hours and 30 hours after dust mite challenge. Histamine sensitivity was assessed 30 hours after challenge (day 5) and the lungs removed for histology. The following results were obtained:
1—DHEA-S reduced the cellular influx of cells into the BALF at 6 hours after allergen challenge by 62% as compared to Pulmicort (FIG. 4.3.4.1).
2—DHEA-S tended to decrease the number of eosinophils 6 hours after allergen challenge, while Pulmicort reduced the number of eosinophils at 24 hours post-allergen (FIG. 4.3.4.1).
3—DHEA-S reduced the number of neutrophils in the BALF 6 hours after allergen challenge by 91% as compared to Puimicort (FIG. 4.3.4.1).
4—DHEA-S decreased epithelial shedding 6 hours after allergen challenge by 54% as compared to Pulmicort (FIG. 4.3.4.1).
5—Both DHEA-S and Pulmicort tended to shift the histamine response curve downward towards pre-allergen challenge values (FIG. 4.3.4.2).
6—DHEA-S and Pulmicort decreased pavementing as compared to saline, tended to decrease necrotizing bronchitis, and tended to increase monocyte phagocytic hyperplasia, but only the effect on pavementing was statistically significant.

These results indicate that both DHEA-S and Pulmicort attenuated inflammation and the decline in pulmonary function normally induced by aeroallergen challenge. In addition, in this study, DHEA-S was equivalent to Pulmicort in its ability to decrease histamine responsiveness and reduce histological indicators of inflammation 24 hours after allergen exposure, and superior in its reductions of inflammatory cells, especially neutrophils, 6 hours after allergen exposure. The differences observed between DHEA-S and Pulmicort in both cell types affected and time course of response suggest the drugs work through dissimilar mechanisms.

### (A) Basal Allergic Responsiveness

### Allergen-Induction of Inflammation.

Examination of the aggregate data (i.e., all groups) revealed a significant increase in eosinophil numbers 6 hours and 24 hours after allergen challenge (P=0.046 and 0.001, respectively). The percent of eosinophils also increased from less than 0.1% before dust mite challenge to 3% twenty-four hours after challenge confirming that the allergen induced migration of eosinophils into the airways.

### Pulmonary Function

The composite histamine dose-response graph for the saline control group exhibited an upward shift of the resistance curve following dust mite challenge (FIG. 4.3.4.2). This provides evidence that dust mite challenge increases bronchial hyperresponsiveness in these animals. These data confirm that the animals in the study responded to the relevant antigen with airway inflammation and altered pulmonary function.

### (B) Effects of PHEA-S & Pulmicort on Inflammation

### Allergen-Induced Inflammation

Six hours following allergen challenge, both saline-treated and Pulmicort-treated groups exhibited a significant increase in the total number of cells in the BALF as compared to pre-challenge value whereas the DHEA-S-treated group did not. See, FIG. 4.3.4.1. Six hours following allergen challenge, the number of eosinophils in BALF of animals treated with DHEA-S tended to be lower than the Pulmicort or saline treated groups. See, FIG. 4.3.4.1. However, variability in the saline treated group and a small sample size kept the difference from being statistically significant. Twenty-four hours after dust mite challenge, both saline and DHEA-S treated groups exhibited significant elevation in the number of eosinophils in BALF relative to the pre-challenge time point. Animals treated with DHEA-S exhibited no allergen-induced increase in neutrophils in BALF at either 6 hours or 24 hours after allergen challenge, in sharp contrast to animals exposed to allergen alone (saline controls). In contrast to the saline control group, the animals treated with Pulmicort did not exhibit a significant increase in the number of eosinophils in the lavage fluid 24 hours after allergen challenge (Pro-dust mite=0.01±0.01×10⁴ eosinophils/ml, 24 hr. post dust mite <=0.7±0.3×10⁴ and 1.5±0.7 eosinophils/ml, Pulmicort and saline, respectively). In addition, the Pulmicort group exhibited an increase in neutrophils 6 hours after dust mite challenge relative to the saline-treated and DHEA-S-treated groups at the same time point, p<0.05. These and the previous results are presented in FIG. 4.3.4.1.

### Histology

Analysis of lung sections indicated a moderate level of pulmonary inflammation in all groups. No differences in the level of monocyte phagocytic hyperplasia, airway and blood vessel cuffing by inflammatory cells, pneumonitis, pneumonia, bronchial associated lymphoidal tissue hyperplasia, septal edema, hemorrhage, alveolar edema, or bronchitis/bronchiolitis was observed, although monocyte phagocytic hyperplasia tended to be higher in the Pulmicort and DHEA-S groups, and bronchitis tended to be lower. Pavementing was significantly higher in the saline group, indicating greater attachment of inflammatory cells to the endothelium, which precedes tissue infiltration.

### (C) DHEA-S & Pulmonary Effects on Pulmonary Function

Examination of resistance responses to low doses of histamine (up to 0.625 mg/ml) indicated that animals treated with Pulmicort or DHEA-S exhibited lower responsiveness than animals treated with saline. As shown in FIG. 4.3.4.2, the composite histamine dose-response graph for the saline control group exhibited an upward shift of the resistance curve following dust mite challenge. There was an increase in the area under the curve of 17.42, which means the animals became more sensitive to histamine following allergen exposure. With both DHEA-S and Pulmicort, there was a downward shift in the histamine-response curve. The resulting change in the area under the curve was −4.52 and −6.70 for DHEA-S and Puimicort, respectively, this means that animals treated with DHEA-S or Pulmicort were less sensitive to histamine following allergen exposure.

### Conclusion

The data are consistent with the previous findings that DHEA-S inhibits allergen-induced eosinophilic and neutrophilic inflammation. The effects produced by DHEA-S were different than those demonstrated by Pulmicort. Unlike Pulmicort, DHEA-S caused a dramatic reduction in neutrophilic inflammation. In addition, DHEA-S's anti-inflammatory effect was maximal at an earlier time point as compared to Pulmicort. The effect on bronchial hyperresponsiveness 24 hours after dust mite was similar for both DHEA-S and Pulmicort. These results indicate that DHEA-S has both the standard properties of the glucocorticoid steroids (e.g., ability to inhibit allergen-induced cosinophilia), and additional properties not shared by the glucocorticoids. In particular, DHEA-S demonstrated the ability to inhibit neutrophilic inflammation, a unique property that would enable DHEA-S to offer increased benefits in both asthma and COPD as compared to glucocorticoids.

### Example 28

### DHEA-S Effect on Bronchial Hyper-responsiveness & Inflammation

This study demonstrates the ability of DHEA-S to reduce bronchial hyperresponsiveness to methacholine challenge in ragweed-sensitized mice. Two groups of mice were sensitized with two intraperitoneal injections and one intranasat administration of ragweed allergen (Ambrosia artemistifolia) on days 0, 4 and 11 respectively. A third group was treated similarly with saline to serve as non-allergic controls. The next two days, the treatment group animals were exposed to DHEA-S delivered as an aerosol (5 mg/ml suspension in saline, 2 ml nebulized) using the whole-body plethysmograph system (Buxco Electronics, Inc., Sharon, Conn.) and a DeVilbiss ultrasonic nebulizer. Twenty-four hours after the last drug exposure, mice were challenged with increasing concentrations of methacholine to determine bronchial hyperresponsiveness and then lavaged to determine relative changes in cell populations in BAL fluid. DHEA-S treatment led to the following changes in the disease status of the animals:
(1) DHEA-S administration two days prior to methacholine challenge resulted in a marked decrease in bronchial hyperresponsiveness. The urea under the curve (AUC) of the methacholine response was reduced 46% with DHEA-S treatment.
(2) DHEA-S twenty-four hours prior to challenge results in a significant decrease in neutrophils (3.2% vs. 13.8%, DHEA-S and Ragweed alone, respectively) and a shift of cellular populations from the allergic profile towards one more similar to the non-allergic mice.

### (A) DHEA-S Effect on Pulmonary Function

The non-allergic saline control mice demonstrated minimal change in enhanced pause (Penh- an index of airway obstruction) in response to methacholine having a mean area under the response curve of 2646.8. The ragweed only group responded in a dose-related fashion with increasing Penh value over the range of methacholine tested and a mean area under the curve of 7488.8. This demonstrates that the sensitization procedure results in an increase in bronchial hyperresponsiveness. Treatment with DHEA-S two days prior to methacholine challenge resulted in a marked decrease in bronchial hyperresponsiveness and a resulting mean area under the curve of 4038.0. These results are shown in FIG. 4.3.5.1.

### (B) DHEA-S Effect on Inflammation

The populations of cells in the non-allergic saline control and allergic ragweed group are strikingly different following methacholine challenge. Ragweed sensitization results in a significant increase in the percent eosinophils, lymphocytes, and neutrophils compared to the saline control mice (P<0.05). Treatment of allergic mice with DHEA-S twenty-four hours prior to challenge results in a shift of cellular populations from the allergic profile towards one more similar to the non-allergic mice. There was also a significant reduction in the percent neutrophils in the DHEA-S group compared to the ragweed alone (3.2% vs. 13.8%, P<0.05). These results are shown in FIG. 4.3.5.2.

### Conclusion

The data in this study demonstrate that treatment with DHEA-S reduced the bronchial hyperresponsiveness in response to methacholine in ragweed-sensitized mice. There was also a reduction in the proportion of inflammatory cells in the BAL, particularly eosinophils and neutrophils, p<0.05, immediately following methacholine challenge.

### Example 29

### DHEA-S Lacks Toxicity Effect

Preliminary studies were conducted to assess the feasibility of a nebulized formulation an acute toxicity of inhaled DHEA-S. Administration of DHEA-S by inhalation did not produce any adverse affects to the respiratory tracts of rats or dogs up to the maximum dose that was feasible based on solubility and exposure time limits. A maximum tolerated dose could not be achieved for either species. Other findings were minor in nature and detailed in the reports that follow. Therefore, nebulization of an aqueous solution was determined to be feasible but not practical due to the inability to achieve a sufficient safety factor to support clinical trials.

The foregoing examples are illustrative of the present invention, but should not to be construed as limiting thereof. The invention is defined by the following claims, with equivalents of the claims to be included therein.

What is claimed is:

1. A pharmaceutical composition, comprising a pharmaceutically or veterinarily acceptable carrier and amounts of the first and second active agents effective to treat a respiratory disease,
(a) the first active agent being dehydroepiandrosterone (DHEA) or dehydroepiandrosterone sulfate (DHEAS); or pharmaceutically or veterinarily acceptable salts thereof; and
(b) the second active agent is selected from the group consisting of atropine, ipratropium bromide and tiotropium bromide.

2. The composition of claim 1, comprising about 0.01 to about 99.9% w/w first and second active agent and/or pharmaceutically or veterinarily acceptable salts thereof.

3. The composition of claim 2, comprising about 1 to about 20% w/w first and second active agents and/or pharmaceutically or veterinarily acceptable salts thereof.

4. The formulation of claim 1, in the form of an oral, nasal, inhalable, or respirable formulation.

5. The formulation of claim 4, which is an aerosol or spray comprising solid powdered particles.

6. The inhalable or respirable formulation of claim 42, comprising particles substantially about 0.05 to about 10μ in size (diameter).

7. The inhalable or respirable formulation of claim 6, comprising particles substantially about 0.1 to about 5μ in size.

8. The inhalable or respirable formulation of claim 7, comprising particles substantially about 1 to about 5μ in size.

9. A method for treating asthma in a subject in need of treatment, comprising simultaneously or separately administering to a subject in need of treatment a therapeutically effective amounts of the first and second active agents of claim 1.

10. The method of claim 9, wherein the first active agent is administered in an amount of about 0.05 to about 2000 mg/kg body weight/day.

11. The method of claim 10, wherein the first active agent is administered in an amount of about 1 to about 500 mg/kg/day.

12. The method of claim 11, wherein the first active agent is administered in an amount of about 2 to about 100 mg/kg/day.

13. The method of claim 9, wherein the subject is a human or a non-human animal.

14. The method of claim 9, further comprising administering to the subject a β-adrenergic agonist selected from ephedrine, isoproterenol, isoetharine, epinephrine, metaproterenol, terbutaline, fenoterol, procaterol, albuterol, salbutamol, pirbuterol, formoterol, biloterol, bambuterol, salmeterot or seretide.

15. The method of claim 9, wherein the first active agent is administered in an amount of about 2 to about 200 mg/kg/day; and the second active agent comprises ipratropium bromide and is administered in therapeutic amounts.

16. The method of claim 9, wherein the first and second active agents are administered by inhalation or respiration into the airways.

17. The method of claim 16, wherein the agents are administered as a liquid or powdered aerosol or spray of particle size substantially about 0.05 to about 10 μm.

18. The method of claim 16, wherein the agents are administered as a liquid or powdered aerosol or spray of particle size substantially about 10 to about 50 μm.

19. The method of claim 16, wherein the first and second agents are administered in the same composition.

20. The method of claim 19, wherein the first and second agents are administered by inhalation or into the respiratory airways.
